(19) United States
(12) Patent Application Publication
(10) Pub. No.: US 2011/0125178 A1
(43) Pub. Date: May 26, 2011

(54) DEVICES, METHODS AND KITS FOR FORMING TRACTS IN TISSUE

(76) Inventors: Michael Drews, Palo Alto, CA (US); D. Bruce Modesitt, San Carlos, CA (US)

(21) Appl. No.: 12/780,768

(22) Filed: May 14, 2010

Related U.S. Application Data
(60) Provisional application No. 61/178,895, filed on May 15, 2009.

(51) Int. Cl. A61B 17/34; A61B 17/00

(52) U.S. Cl. 606/185; 606/213

(57) ABSTRACT

Described here are devices, methods, and kits for forming one or more tracts in tissue. The tracts may be formed in any suitable or desirable tissue, and may seal relatively quickly without the need for a supplemental closure device. In some variations, the methods may comprise advancing a tissue-piercing member along a predetermined path of a tract-forming device to form one or more tracts in tissue (e.g., a vessel wall). The tract or tracts may, for example, provide access for one or more tools.

### CROSS-REFERENCE TO RELATED APPLICATIONS

This application claims the benefit of U.S. Provisional Application No. 61/178,895, filed on May 15, 2009, the disclosure of which is incorporated herein by reference in its entirety.

### TECHNICAL FIELD

Described here are devices, methods, and kits for forming one or more tracts in tissue. More specifically, described here are devices, methods, and kits for forming one or more tracts in tissue using a tissue-piercing member that moves along a path predetermined by one or more features (e.g., grooves) of a device.

### BACKGROUND

A number of devices and methods have previously been described for forming tracts in or through tissue. For example, devices and methods for forming tracts in tissue are described in U.S. patent application Ser. No. 10/844,247 (published as US 2005/0267520 A1); U.S. patent application Ser. No. 11/544,196 (published as US 2007/0027455 A1); U.S. patent application Ser. No. 11/545,272 (published as US 2007/0032804 A1); U.S. patent application Ser. No. 11/544,365 (published as US 2007/0032803 A1); U.S. patent application Ser. No. 11/544,177 (published as US 2007/0027454 A1); U.S. patent application Ser. No. 11/544,149 (published as US 2007/0032802 A1); U.S. patent application Ser. No. 10/888,682 (published as US 2006/0009802 A1); U.S. patent application Ser. No. 11/432,982 (published as US 2006/0271078 A1); U.S. patent application Ser. No. 11/544,317 (published as US 2007/0106246 A1); U.S. patent application Ser. No. 11/788,509 (published as US 2007/0255313 A1); U.S. patent application Ser. No. 11/873,957 (published as US 2009/0105744 A1); U.S. patent application Ser. No. 12/507,038 (published as US 2010/0016786 A1); and U.S. patent application Ser. No. 12/507,043 (published as US 2010/0016810 A1), all of which are incorporated herein by reference in their entirety. In general, the tracts described in these applications may self-seal or seal after they have been formed, with minimal or no need for supplemental closure devices or techniques. These tracts may be quite useful in providing access to a tissue location (e.g., an organ lumen) so that one or more tools may be advanced through the tracts, and one or more procedures may be performed. Given the tremendous applicability of such methods, additional devices and methods for forming tracts in tissue would be desirable.

### BRIEF SUMMARY

Devices, methods, and kits for forming one or more tracts in tissue are described herein. Generally, a device for forming a tract in tissue may be used to advance a tissue- piercing member along a predetermined path. The tissue-piercing member may enter tissue at some point prior to, during, or after its advancement along the predetermined path. As the tissue-piercing member is being advanced through the tissue, it may form a tract in the tissue. The tract may then be used, for example, to advance one or more tools or other devices to a target site.

In some variations, a device for forming one or more tracts in tissue may comprise a housing and a tissue-piercing member coupled to the housing, where the tissue-piercing member is movable relative to the housing along a path predetermined by one or more features (e.g., grooves) in the housing. The advancement of the tissue-piercing member along the predetermined path may allow for relatively controlled formation of a tract in tissue (e.g., requiring little manipulation of the device by the operator). For example, the presence of the predetermined path may allow for the tissue-piercing member to be well-positioned relative to the tissue prior to piercing the tissue.

Devices, methods, and kits described here may provide for reproducible tract formation. For example, some variations of devices described here may be used to form substantially similar tracts through two different vessel walls, or through two different locations in the same vessel wall. As such, results may be relatively easy to predict in advance of a procedure. Moreover, there may be a low likelihood for error in a given procedure, as a result of this predictability. In some variations, the path of a tissue-piercing member may be selected based on, for example, the thickness, mechanical characteristics, and/or biological characteristics of the tissue in which a tract is to be formed.

Certain variations of devices described here may be relatively easy to use. For example, a device may be used to form a tract in tissue without requiring significant torquing or other manipulation by the operator. In some variations, a device described here may be successfully used by an operator who has had relatively little training, and/or who has had limited experience in the particular procedure at hand. For example, an operator who has had limited experience in forming an arteriotomy may be able to use the device to successfully form an arteriotomy. Devices described here may be unlikely to be misused. For example, in some variations a device may be actuated by simply pushing a button or operating a slide actuator. Such simplicity in actuation may limit the likelihood of confusion during operation of the device, and may also allow for operation of the device by operators having different skill levels. As an example, in some cases a device may be capable of operation by both a highly skilled operator and a relatively inexperienced operator. Additionally, devices that are relatively easy to use may be safer than other devices because, for example, they may result in a reduced likelihood of operator error.

In some variations, an operator may be able to operate a device described here at a distance from the body of the person undergoing the procedure, while having localized control over the advancement and/or positioning of the tissue-piercing member. For example, the operator may be able to actuate the device by manipulating a tensioning member that is coupled to the device, and/or by pressing or actuating one or more buttons, switches, etc., without significantly manipulating the body of the device itself. This, in turn, may prevent the operator from having to advance a long tissue-piercing member down a long tissue-piercing member port, in a tortuous or bending path, to reach a target site. Such advancement can result in high frictional forces between the tissue-piercing member and the tissue-piercing member port, thereby requiring that the operator exert a substantial force to overcome the high frictional forces and advance or withdraw the tissue- piercing member. When the operator exerts the substantial force, the device and/or surrounding tissue may become damaged, and/or the tissue-piercing member may be over-advanced (e.g., because of initial stiction that then releases unpredictably). Devices described here may avoid these problems by substantially eliminating the need to push and/or pull a tissue-piercing member along their length. Moreover, a tissue-piercing member may be actuated in tortuous positions without requiring that a bulky tissue-piercing member shaft maneuver the same tortuous path.

In certain variations, devices, methods and/or kits described here may be used to form a self-sealing tissue tract. A self-sealing tissue tract does not need interventional devices or methods to help it seal—by definition, it seals by itself. For example, a self-sealing tissue tract does not need a plug, energy, sealants, clips, sutures, or the like to help it seal. Rather, a self-sealing tissue tract may seal when opposing tissue portions along the tract contact each other and form a seal. This may occur, for example, when the angle of the tract relative to the tissue wall is relatively shallow, which may result in the tract having a relatively long length and/or high surface area. Blood pressure may cause the tissue portions to come into contact with each other and natural clotting factors and the like may cause them to form a seal. Of course, it should be understood that, as described later herein, manual pressure or compression may be applied to a self-sealing tract to expedite its sealing, without affecting the self-sealing nature of the tract.

In some variations, a device may comprise a housing comprising at least one groove, and at least one tissue-piercing member (i.e., one tissue piercing member or a plurality of tissue-piercing members, such as two, three, four or five tissue-piercing members) coupled to the housing. The tissue-piercing member may comprise a distal end having a tissue-piercing tip, and may be movable relative to the housing along a path predetermined by the configuration of the groove.

In certain variations, the device may further comprise at least one protruding member (e.g., at least one pin) coupled to the tissue-piercing member and slidably disposed within the groove. In some variations, the device may comprise a tissue-piercing member directing device (e.g., that couples the tissue-piercing member to the housing). In some such variations in which the device also comprises at least one protruding member, the protruding member and the tissue-piercing member may both be coupled to the tissue-piercing member directing device. In certain variations, the tissue-piercing member directing device may comprise at least one portion that is slidably disposed within the groove. For example, the tissue-piercing member directing device may comprise a body and at least one protruding member (e.g., at least one pin) that protrudes from the body and that is slidably disposed within the groove. In some variations, the body of the tissue-piercing member directing device may define an aperture (e.g., a channel) through which the tissue-piercing member passes.

In certain variations, the device may comprise at least one tensioning member, such as a cable. In some variations in which the device comprises a protruding member, the tensioning member may be coupled (e.g., fused) to the protruding member. Alternatively or additionally, the protruding member may define an aperture therethrough. The tensioning member may pass through the aperture and in some cases, the tensioning member may be coupled to the protruding member within the aperture. In certain variations, the device may comprise a rotatable member coupled to or integral with the housing, and the tensioning member may be coupled to the rotatable member. The rotatable member may, for example, comprise a pulley, and the tensioning member may be wound around the pulley. In some variations, the device may comprise a protruding member (e.g., a post) coupled to or integral with the housing, where the tensioning member is coupled to (e.g., wound around) the protruding member. In certain variations, proximal translation of the tensioning member may result in distal translation of the tissue-piercing member.

In some variations, the tissue-piercing member may be coupled to the housing at a pivoting point. In certain variations, at least a portion of the tissue-piercing member may be disposed within a tubular member that is coupled to the housing at a pivoting point. The device may comprise at least one tensioning member that is coupled to both the housing and the tissue-piercing member, and that is manipulatable to cause the tissue-piercing member to pivot about the pivoting point. The tissue-piercing member may comprise a needle. The tissue-piercing member may comprise at least one shape-memory material and/or at least one superelastic material.

In some variations, the device may comprise a tubular member. The tubular member may be articulatable relative to the housing and/or may be coupled to the housing by at least one flexible member (e.g., at least one hinge). In certain variations, a portion of the tissue-piercing member may be disposed within the tubular member. In some variations, the tubular member may be coupled to the tissue-piercing member. In some such variations, the tubular member may couple a port (e.g., a port on the housing) to the tissue-piercing member such that the port and the tissue-piercing member are in fluid communication with each other. In certain variations, the housing may comprise at least two grooves therein, and the tubular member may be positioned between at least two pins that are slidably disposed within the grooves.

The housing may comprise at least one indented portion. In some variations, the device may comprise a vacuum port located in the indented portion. Alternatively or additionally, the housing may comprise at least one hook or barb protruding from the indented portion, and/or may have a rough surface or coating in the indented portion.

The device may comprise at least one sensor. For example, the device may comprise one or more temperature sensors, pressure sensors, and/or blood flow sensors (e.g., Doppler).

In some variations, a method for forming a tract in tissue may comprise positioning a tract-forming device adjacent to the tissue, the tract-forming device comprising a housing and a tissue-piercing member coupled to the housing, and advancing the tissue-piercing member along a predetermined path (e.g., a curvilinear path) defined by at least one groove in the housing. The tract-forming device may comprise a tensioning member coupled to the tissue-piercing member and the housing, and the method may comprise manipulating the tensioning member to advance the tissue-piercing member along the predetermined path. In some variations, the tensioning member may be manipulated by proximally withdrawing the tensioning member. In certain variations, the tissue-piercing member may be at least partially disposed within a tubular member that is coupled to the housing, and the method may comprise manipulating the tubular member to advance the tissue-piercing member along the predetermined path. In some variations, the method may comprise advancing the tissue-piercing member into the tissue.

In certain variations, a method for forming a tract in a vessel wall (e.g., an arterial wall) may comprise positioning a tract-forming device adjacent the vessel wall, and advancing a tissue-piercing member along a predetermined path of the tract-forming device to form a tract in the vessel wall, where a portion (e.g., a minority or a majority) of the tract traverses the vessel wall substantially parallel to a longitudinal axis of the vessel wall.

In certain variations, a method for forming a tract in a vessel wall (e.g., an artery wall) may comprise positioning a tract-forming device adjacent the vessel wall, and advancing at least one tissue-piercing member along a predetermined path of the tract-forming device to form a tract in the vessel wall, where the tract forms an angle of less than or equal to about 30° (e.g., less than or equal to about 19°, less than or equal to about 15° , less than or equal to about 10°, less than or equal to about 5°, from about 1° to about 30°, from about 1° to about 19°, from about 1° to about 15°, from about 1° to about 10°, from about 1° to about 5°, from about 5° to about 15°, from about 5° to about 10°) with respect to a longitudinal axis of the vessel wall.

The tract-forming device may comprise a housing that remains in substantially the same position while the tissue-piercing member forms the tract in the vessel wall. In some variations, the tract-forming device may comprise a housing and a tensioning member coupled to both the tissue-piercing member and the housing. In some such variations, the method may comprise manipulating the tensioning member to advance the tissue-piercing member along the predetermined path. The tensioning member may be manipulated by proximally withdrawing the tensioning member.

The tissue-piercing member may enter the tissue at a first location, and exit the tissue at a second location, and the length between the first and second locations may be greater than the thickness of the tissue or the tissue wall (e.g., vessel wall). In certain variations, the length of the tract may be greater than the thickness of the tissue or the tissue wall (e.g., vessel wall). In some variations, the method may comprise advancing one or more tools and/or closure devices into and/or through the tract. For example, in certain variations, a sheath (e.g., an introducer sheath) may be advanced into and/or through the tract. The sheath may, for example, be used to expand the tract for performance of a procedure therethrough. After the procedure has been completed, the sheath may be withdrawn and the tract may seal (e.g., self-seal). In some variations, the method may comprise applying pressure to the tract.

Certain variations of methods described here may comprise withdrawing the tissue-piercing member from the tissue or tissue wall (e.g., vessel wall). The tract may self-seal after the tissue-piercing member has been withdrawn. As described above, a self-sealing tissue tract does not need interventional devices or methods to help it seal—by definition, it seals by itself. For example, a self-sealing tissue tract does not need a plug, energy, sealants, clips, sutures, or the like to help it seal.

Tracts formed here may self-seal relatively quickly. For example, the tracts may seal within 15 minutes or less (e.g., within 12 minutes or less, within 10 minutes or less, within 9 minutes or less, within 6 minutes or less, within 5 minutes or less, within 3 minutes or less, within 1 minute or less, etc.). Of course, if so desired, one or more supplemental closure devices may be used in conjunction with the described devices and methods.

The methods may comprise sensing at least one of temperature, pressure, and blood flow. In some variations, a method may additionally comprise applying a vacuum to tissue and/or clamping tissue. In certain variations, a method may comprise advancing a tissue-piercing member into tissue after applying a vacuum to the tissue and/or clamping the tissue. Some variations of methods described here may also comprise clamping or otherwise isolating tissue, and positioning the tissue for relatively easy advancement of a tissue-piercing member therethrough, to form a tract in at least a portion of the tissue. Methods for applying a vacuum or suction to tissue, as well as clamping methods and other tissue-positioning or isolation methods, are described, for example, in U.S. patent application Ser. No. 12/507,038 (published as US 2010/0016786 A1) and U.S. patent application Ser. No. 12/507,043 (published as US 2010/0016810 A1), both of which were previously incorporated herein by reference in their entirety.

In some variations, a method for forming a tract in tissue (e.g., a vessel wall) of a subject may comprise forming a single tract in the tissue. The single tract may, for example, be self-sealing. As described above, a self-sealing tissue tract does not need interventional devices or methods to help it seal—by definition, it seals by itself. For example, a self-sealing tissue tract does not need a plug, energy, sealants, clips, sutures, or the like to help it seal. The single tract may be formed, for example, by advancing only one tissue-piercing member through the tissue. This may, for example, result in minimal stress on the tissue. Moreover, the tissue may recover relatively quickly, thereby resulting in relatively short procedure time. In certain variations, the single tract may be used to provide access to a region defined by tissue, such as a lumen defined by a vessel wall. In some variations, a method for forming a tract (e.g., a self-sealing tract) in tissue (e.g., a vessel wall) of a subject may comprise forming a tract in the tissue by advancing at least one tissue-piercing member through at least a portion of tissue, where formation of the tract requires advancement only of the tissue-piercing member or members through at least the portion of the tissue.

The tissue-piercing member may be, for example, a needle, such as a hollow needle or a solid needle. The needle may have any suitable tip having any suitable shape. For example, the tip may be conical, offset conical, blunt, sharpened or pointed, beveled, non-beveled, etc.

Tracts formed using the devices, methods, and/or kits described here may be formed in any suitable or desirable tissue. For example, the tissue may be an organ of any of the body systems (e.g., the cardiovascular system, the digestive system, the respiratory system, the excretory system, the reproductive system, the nervous system, etc.). In certain variations, the tissue may be an organ of the cardiovascular system, such as the heart or an artery. In other variations, the tissue may be an organ of the digestive system, such as the stomach or intestines. In some variations, the tissue may be tissue of a vessel wall (e.g., an arterial wall). The devices and methods may be used in any tissue for which their use is appropriate.

### BRIEF DESCRIPTION OF THE DRAWINGS

FIGS. 1A-1F are side cross-sectional views depicting the deployment of a tissue-piercing member from a variation of a device for forming one or more tracts in tissue; FIGS. 1G-1J show the device of FIGS. 1A-1F at different points in time during the advancement of the tissue-piercing member; and FIGS. 1K-1Q depict the formation of a tract in tissue using the device of FIGS. 1A-1J.

FIGS. 1R-1T depict different variations of tracts in tissue.

FIG. 1U is a side cross-sectional view of another variation of a device for forming one or more tracts in tissue.

FIG. 1V is an illustrative cross-sectional depiction of a variation of a self- sealing tract through a vessel wall.

FIGS. 2A-2G illustrate a variation of a method for forming one or more tracts in tissue using the device of FIGS. 1A-1J.

FIGS. 3A-3C depict different variations of devices for forming one or more tracts in tissue.

FIGS. 4A-4D are partial cross-sectional views of a device for forming one or more tracts in tissue, illustrating the deployment of a tissue-piercing member from the device.

FIGS. 5A and 5B are partial cross-sectional views depicting additional variations of a device and method for forming one or more tracts in tissue.

FIG. 5C is a partial cross-sectional view of a variation of a device for forming one or more tracts in tissue.

FIGS. 6A and 6B depict variations of a device and a method for forming one or more tracts in tissue.

FIGS. 7A-7C illustrate the deployment of a tissue-piercing member from another variation of a device for forming one or more tracts in tissue.

FIGS. 8A and 8B depict an additional variation of a device for forming one or more tracts in tissue, and a related method.

FIG. 9A is an illustrative depiction of the distal end of an exemplary device that may be used to form one or more tracts in tissue, and FIGS. 9B-9N depict an illustrative method for forming a tract in tissue using the device of FIG. 9A, with FIGS. 9H and 9I specifically depicting suitable distal expandable features of a guidewire for use with methods described herein.

FIG. 10A is an overview illustration of how devices described herein may be advanced through a natural body orifice and used to form a tract in tissue, in this case, the stomach.

FIGS. 10B and 10C provide illustrative variations of fluid delivery and collection configurations.

FIGS. 11A-11I depict an illustrative method for forming a tract in or through stomach tissue.

FIGS. 12A-12D depict an illustrative method of accessing the pericardial space in connection with the methods described herein.

FIGS. 13A-13K depict an illustrative method for forming a tract in heart tissue.

### DETAILED DESCRIPTION

Described here are devices, methods and kits for forming tracts in tissue. For example, the devices, methods and kits described here may be used to form a tract through a tissue wall, such as a vessel wall. In general, the tracts may be formed relatively easily, and in a controlled manner. The formation of a particular tract may also be reproducible. As a result, the devices, methods and/or kits described here may provide a procedure with enhanced predictability. The devices, methods and kits described here may allow for relatively accurate, easy, and efficient tract formation. In certain variations, the devices, methods and/or kits described here may be used by an operator with limited experience or even no experience (e.g., for training). Because the devices, methods and/or kits may be relatively easy and simple to use, a relatively inexperienced operator may be able to successfully perform a procedure using the devices, methods and/or kits.

In some cases, tracts formed using the devices, methods and/or kits described here may be capable of self-sealing with minimal or no additional sealing efforts. As described above, a self-sealing tissue tract does not need interventional devices or methods to help it seal—by definition, it seals by itself. For example, a self-sealing tissue tract does not need a plug, energy, sealants, clips, sutures, or the like to help it seal. It should be understood, however, that the devices, methods and kits described here may be complemented by the use of one or more additional closure mechanisms or techniques (e.g., closure devices, delivery of energy, application of pressure, etc.). For example, in some variations, compression may be applied to achieve hemostasis.

In certain variations, devices and/or methods described here may be used to form a single tract in tissue (e.g., where the single tract is self-sealing).

The devices, methods and kits described here may be used with any tissue in which it is desired to form one or more tracts. For example, the tissue may be an organ, such as an organ of any of the body systems (e.g., the cardiovascular system, the respiratory system, the excretory system, the digestive system, the reproductive system, the nervous system, etc.). In some variations, the tissue may be an organ of the digestive system, such as the stomach or intestines. In other variations, the devices, methods and kits may be used with tissue of the cardiovascular system, such as the vasculature (e.g., an artery) or the heart. As an example, one or more tracts may be formed through a muscular wall and/or septum of a heart to access the left ventricle, the aorta, the aortic valve, the mitral valve, the aortic arch, etc. For example, a tissue-piercing member may be used to form a tract from a peripheral surface of a heart, through a muscular wall of the heart, and into a septum of the heart. In certain variations, a tissue-piercing member may be used to form a transapical tract into a heart. In some variations, the tissue may be an artery, and the methods may be used in conjunction with performing an arterial puncture. In certain variations, the tissue may be accessed through a natural orifice (e.g., to perform natural orifice translumenal endoscopic surgery, or “NOTES”). The tissue may be, for example, tissue of the reproductive system, excretory system, digestive system, or the like. Of course, it should be understood that methods of forming multiple tracts in tissue, whether through similar or different tissue, are also contemplated.

While the devices, methods, and kits described herein are described with respect to tissue tract formation, some variations of the devices, methods, and/or kits may alternatively or additionally be used for one or more other purposes. For example, they may be used to deliver one or more diagnostic and/or therapeutic agents (e.g., drugs) to tissue.

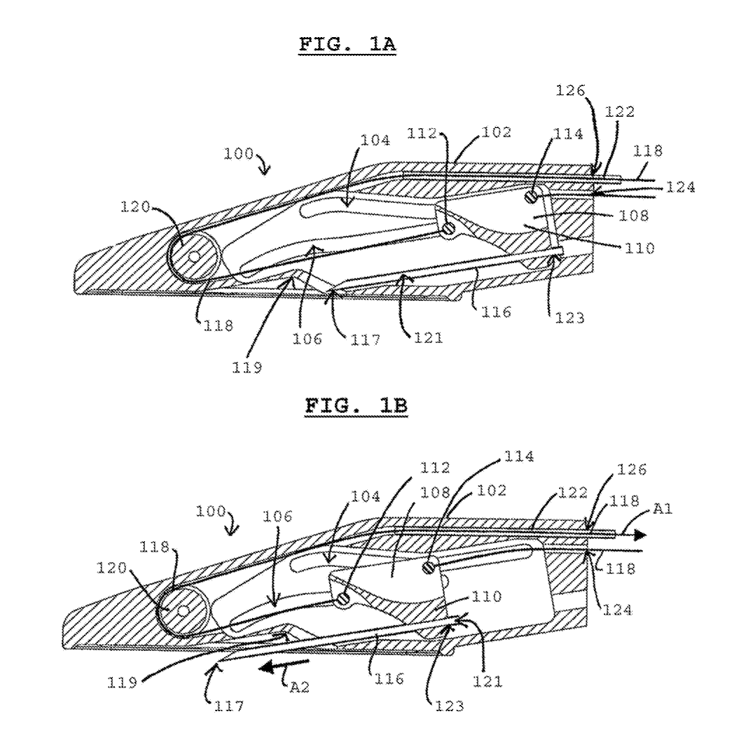

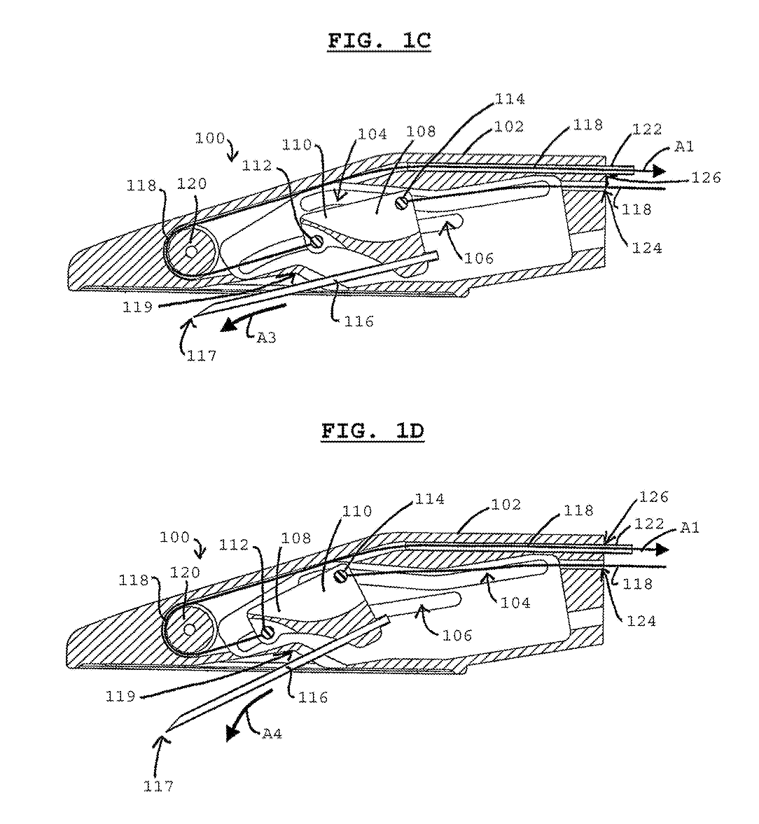

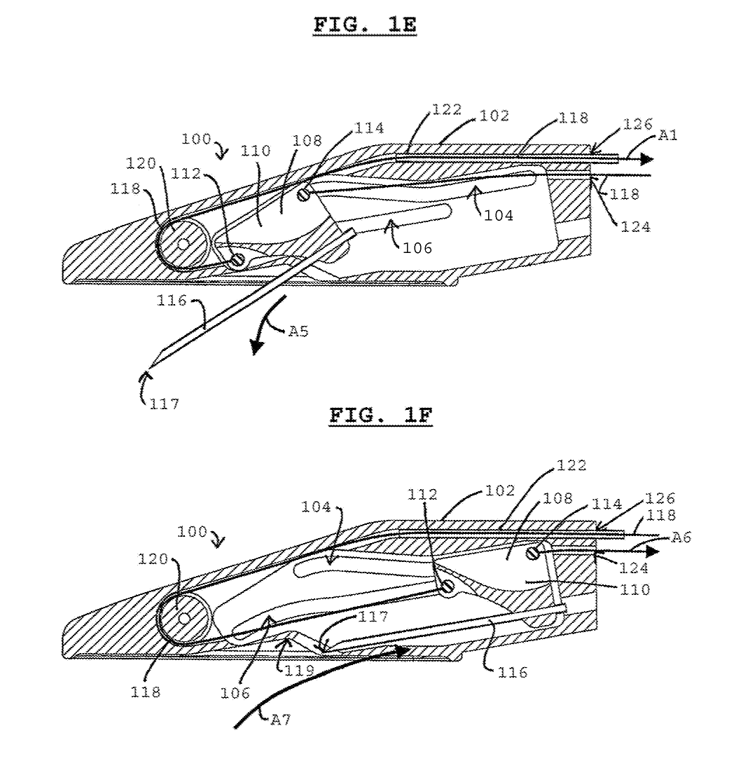

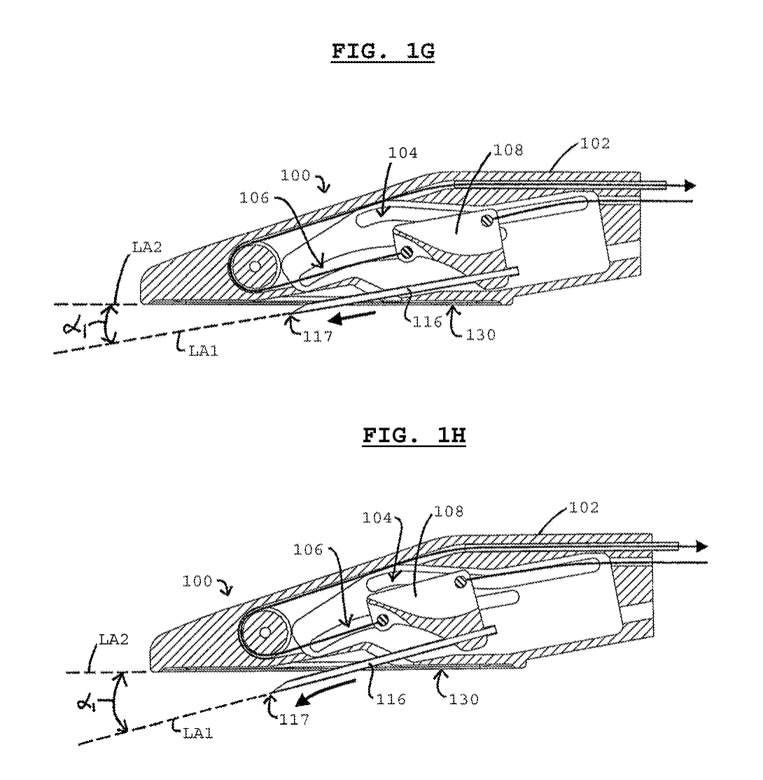

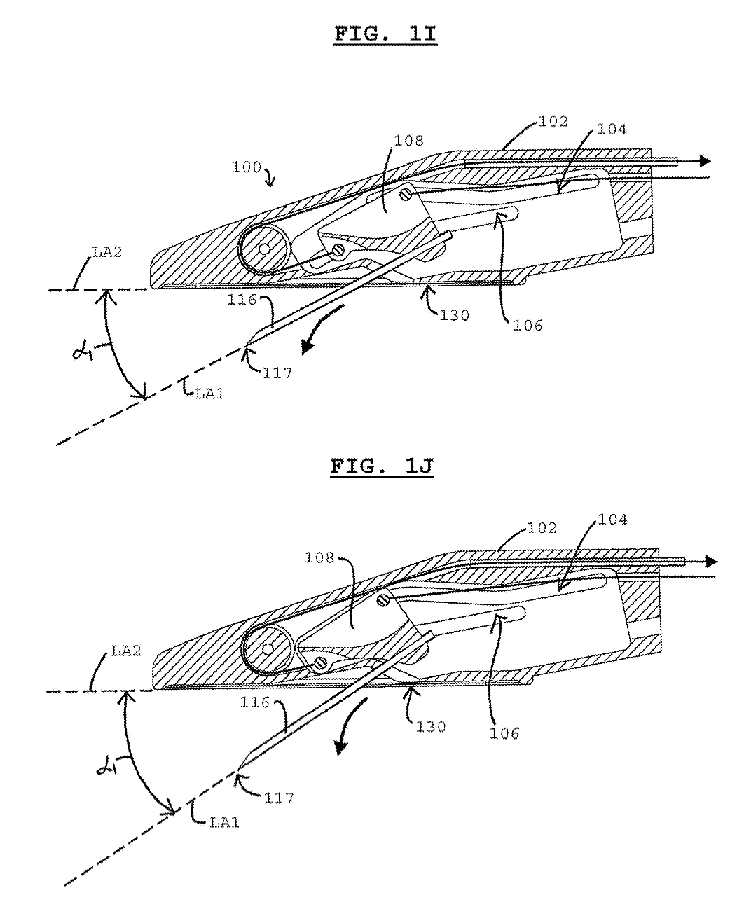

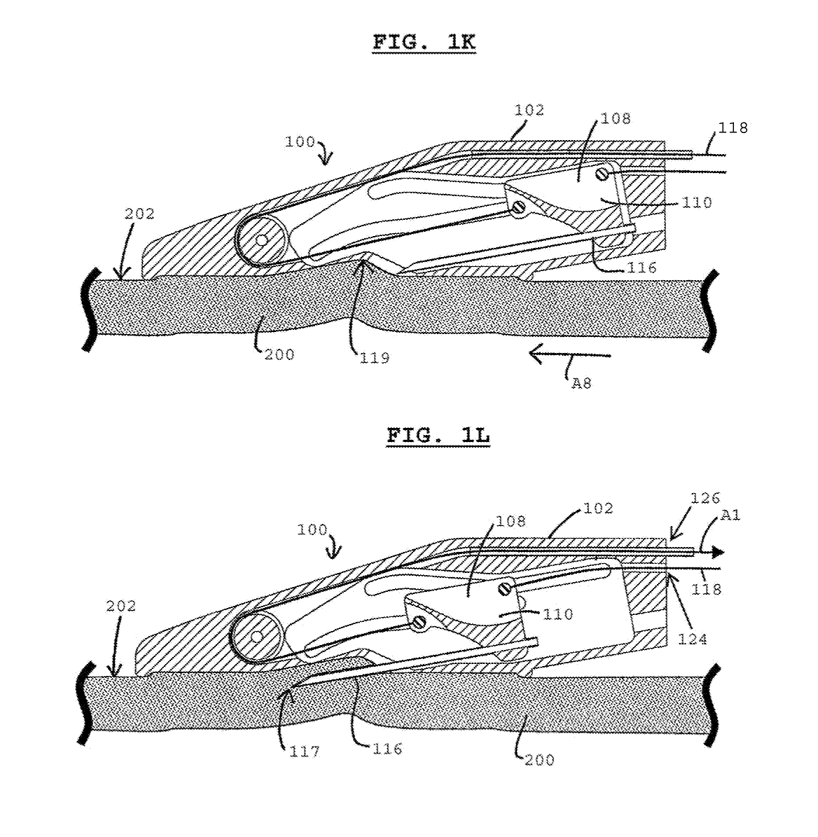

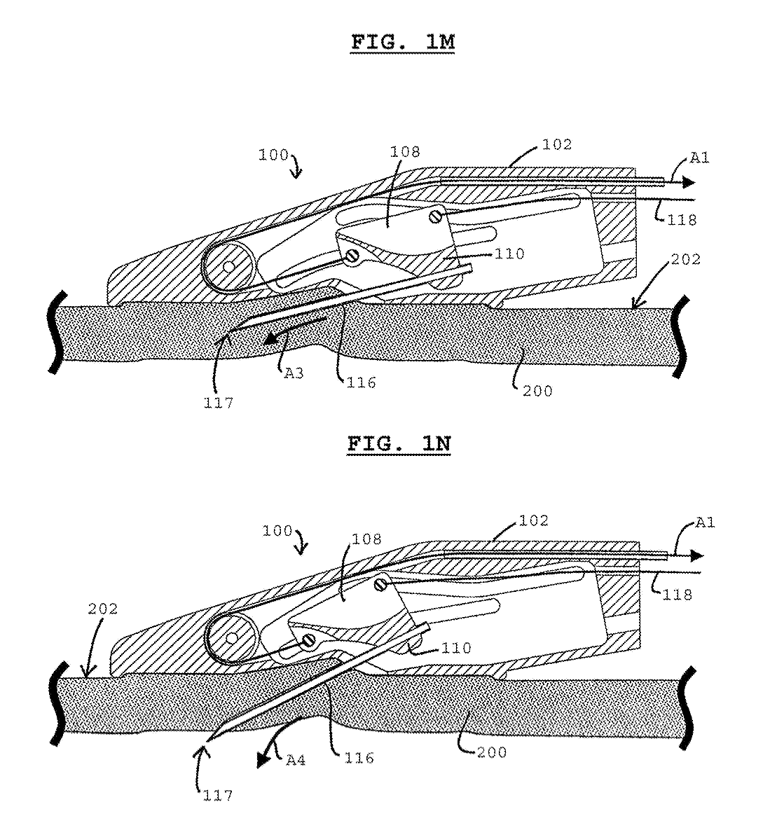

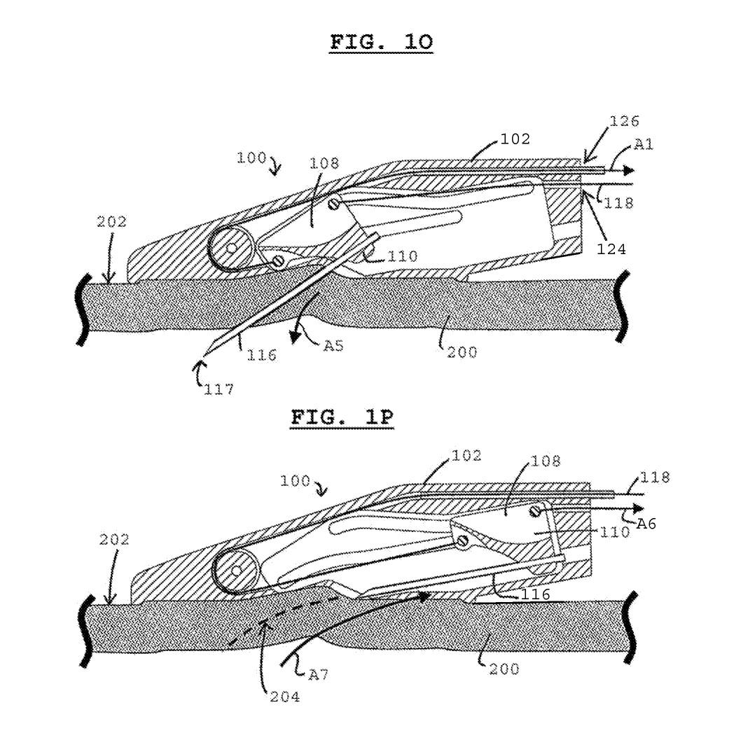

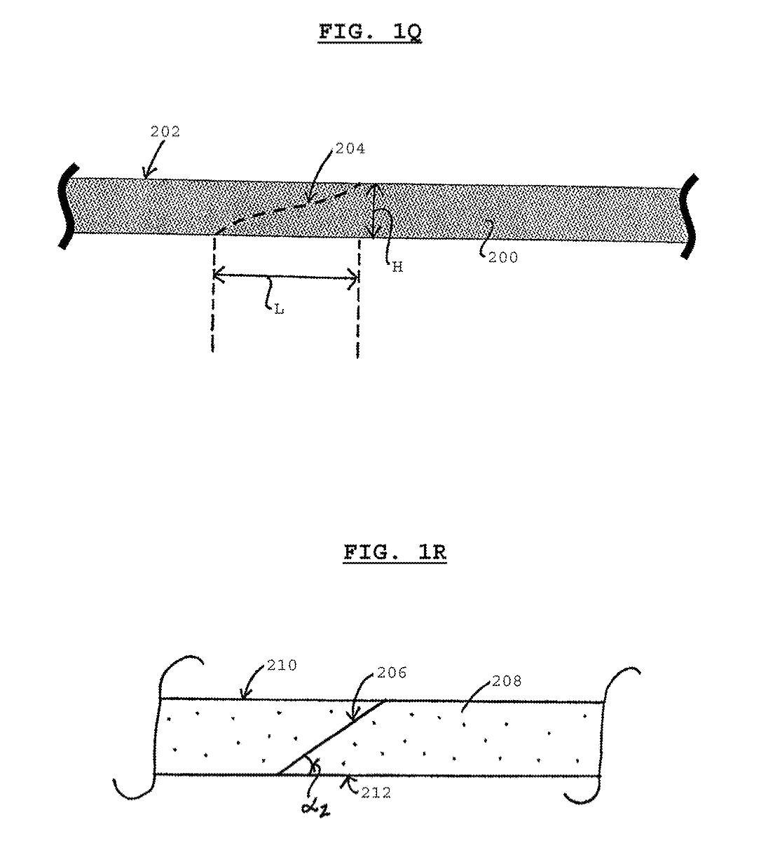

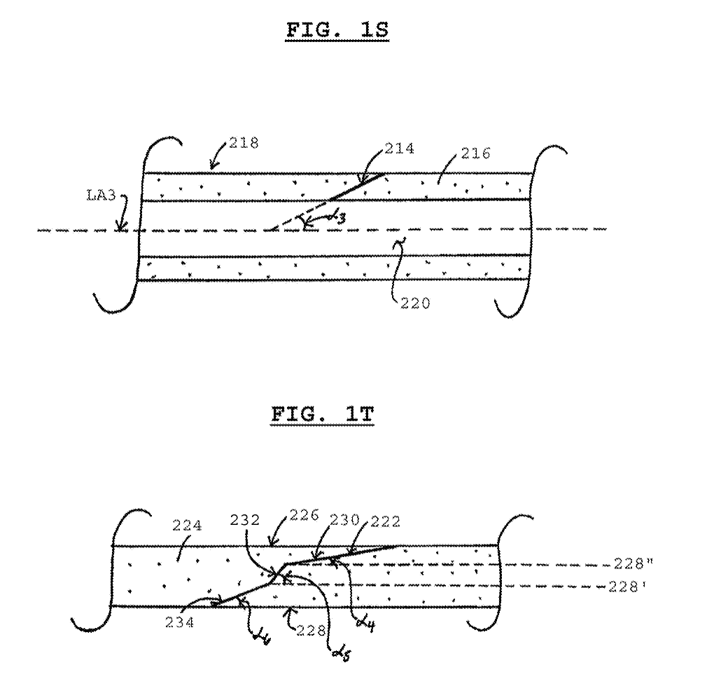

FIGS. 1A-1F depict an exemplary variation of a device (100) for forming one or more tracts in tissue, and its operation. As shown in FIG. 1A, device (100) comprises a housing (102) having two grooves (104) and (106) therein. Housing (102) has an indented portion (119) that may be used, for example, to help position tissue for tissue-piercing, as described in further detail below. Device (100) also comprises a tissue-piercing member directing device (108) comprising a body (110) and two protruding members (as shown, pins (112) and (114)) protruding from the body. Pins (112) and (114) are slidably disposed within grooves (104) and (106). Tissue-piercing member directing device (108) is coupled to a tissue-piercing member (116) having a tissue-piercing tip (117) and a lumen (121). As shown, tissue-piercing member (116) passes through an aperture (here, a channel (123)) in body (110) of tissue-piercing member directing device (108). However, in some cases, a tissue-piercing member may be coupled to a tissue-piercing member directing device in a different way. For example, a tissue-piercing member may be attached (e.g., welded) to an outer surface of the tissue-piercing member directing device. In some variations, a tissue-piercing member may be integral with a tissue-piercing member directing device.

In this variation, tissue-piercing member directing device (108) is also coupled to a tensioning member (118). Tensioning member (118) may, for example, extend through an aperture (not shown) in body (110) of tissue-piercing member directing device (108). Generally, tensioning member (118) may be coupled to body (110). For example, the tensioning member may be welded, fused, adhered, molded, or over-molded, or attached or connected in any other suitable manner, to the body within the aperture. Because of this coupling, manipulation of the tensioning member can result in a corresponding movement by the tissue-piercing member directing device. While one aperture has been described, in some cases a tensioning member may extend through multiple (i.e., at least two) apertures in a body of a tissue-piercing member directing device. In certain variations, tensioning member (118) may alternatively or additionally be coupled to one or both of pins (112) and (114). As an example, the tensioning member may be wound around at least one of the pins. In some variations, the tensioning member may be fused to at least one of the pins, and/or may pass through one or more apertures in at least one of the pins.

In certain variations, a device may comprise multiple tensioning members, such as two, three, four, or five tensioning members. For example, FIG. 1U shows a device (180) for forming one or more tracts in tissue. The device comprises a tissue-piercing member directing device (182) having a body (184), two pins (186) and (188) protruding from the body, and tensioning members (190) and (192), one connected to each pin. One, both, or neither of the tensioning members may pass through one or more apertures (not shown) in body (184) of tissue-piercing member directing device (182). Other arrangements of tensioning members may also be used.

While pins (112), (114), (186), and (188) are shown, any appropriate protruding members or combinations of different protruding members (e.g., posts, etc.) may be used in any of the devices described herein. Moreover, devices may comprise any appropriate number or combination of protruding members, and in some cases a protruding member may be fixed to a device (e.g., so that the protruding member is not slidable within a track or groove of the device). In devices comprising multiple protruding members, the protruding members may have the same form, size, and/or shape, or may be different in at least one of these respects. In certain variations, protruding members may be individually actuated. In some variations, protruding members may be spring-loaded. This may, for example, allow the protruding members to be able to follow a groove under spring tension without the need for a tensioning member. Alternatively or additionally, using spring-loaded protruding members may help to accommodate for misalignments within the grooves and/or may prevent binding during actuation.

As shown in FIG. 1A, device (100) further comprises a rotatable member, in the form of a pulley (120) that is coupled to housing (102). Tensioning member (118) is wound around pulley (120) and routed through a tubular member (122). Thus, tensioning member (118) enters housing (102) at a first location (124), couples to tissue-piercing member directing device (108), winds around pulley (120), passes through tubular member (122), and exits housing (102) at a second location (126). While tensioning member (118) is routed through a tubular member (122), some variations of devices may not include such a routing tubular member, or may include multiple such routing tubular members (e.g., positioned in different sections of the device). In certain variations, a device may include one or more other components that may be used to help route a tensioning member. In such variations, the device may or may not also include one or more routing tubular members.

Because tensioning member (118) winds around pulley (120), pulley (120) may help to re-direct tension in tensioning member (118). While device (100) comprises pulley (120), some variations of devices may alternatively or additionally comprise one or more other types of components that may be used to re-direct tension in a tensioning member. As an example, a device may comprise a tensioning member wound around a nub or a post (e.g., embedded in plastic, to reduce friction between the nub or post and the tensioning member). As another example, in certain variations a device may comprise a lever that may be used to re-direct tension in a tensioning member. Other appropriate components may also be used.

Tensioning member (118) may be any suitable member to which tension may be applied, and/or any suitable member that may be used to transmit tension. Non-limiting examples of suitable tensioning members include monofilament or multifilament (e.g., braided) sutures or strings, braided metallic cable, and flat bands, strips, or belts comprising one or more metals, plastics, and/or fiber-reinforced polymers.

Referring now to FIG. 1B, when the portion of tensioning member (118) exiting housing (102) at second location (126) is pulled proximally (in the direction of arrow (A1)), tissue-piercing member directing device (108) advances distally along a path predetermined by grooves (104) and (106). Because tissue-piercing member (116) is coupled to tissue-piercing member directing device (108), tissue-piercing member (116) advances along with tissue-piercing member directing device (108). As a result, tissue-piercing member (116) advances in the general direction of arrow (A2). However, the pathway of the tissue-piercing member's advancement may change according to the configuration of grooves (104) and (106). For example, when one or both of the grooves curve, tissue-piercing member directing device (108) may change its direction of advancement as it traverses the curves.

Of course, while FIG. 1B shows grooves having a particular configuration and shape, any other suitable configurations and shapes may be used. Moreover, a device may have any appropriate number of grooves. For example, a device may have just one groove, or may have more than two grooves. The grooves may have any suitable dimensions. In variations in which a device includes at least two grooves, at least two of the grooves may have the same size and/or shape, or all of the grooves may have different sizes and/or shapes. Grooves may be curved, angular, straight, etc. In some variations, a groove may comprise a distinct track or slot, or may have a hemispherical cross-sectional shape. The size, shape, and configuration of a groove may be selected, for example, based on the desired path of advancement of the tissue-piercing member through tissue.

Additionally, while tissue-piercing member directing device (108) advances via the slidable movement of pins within grooves, any other suitable mechanism may be used. As an example, instead of having grooves, a tissue-piercing member directing device may include a housing having one or more rails, and may include features that are adapted to slidably advance along the rails. As another example, instead of (or in addition to) having pins, a tissue-piercing member directing device may include rollers or bearings that are configured to roll along a groove or track in a housing of the device.

Referring now to FIG. 1C, as the operator continues to pull the portion of tensioning member (118) in the direction of arrow (A1), tissue-piercing member directing device (108) changes its orientation as a result of the curvature of grooves (104) and (106). This causes tissue-piercing member (116) to become redirected, such that it now advances distally in the direction of arrow (A3). Similarly, as tissue-piercing member (116) continues to be advanced, it becomes redirected again, so that it advances in the direction of arrow (A4), as shown in FIG. 1D. Referring now to FIG. 1E, tissue-piercing member (116) then becomes redirected once more, such that it is advanced in the direction of arrow (A5). Of course, FIGS. 1B-1E show just one example of various redirections and advancements of a tissue-piercing member, and any combination that is appropriate may be used. The particular combination of advancements and redirections for a tissue- piercing member may be selected, for example, based on the targeted anatomy, other physiological characteristics of the subject (e.g., estimated blood clotting time), the shape of a tool to be introduced through the tract that is formed, etc.

As shown in FIG. 1F, in some variations, tissue-piercing member (116) may also be retractable, such that it can be proximally withdrawn back into housing (102) of device (100). More specifically, by pulling on the portion of tensioning member (118) exiting housing (102) at first location (124) (in the direction of arrow (A6)), tissue-piercing member (116) may be withdrawn back into housing (102) in the direction of arrow (A7).

Referring now to FIGS. 1G-1I, as tissue-piercing member (116) is advanced from device (100), the position of the tissue-piercing member relative to the device changes. This occurs as a result of tissue-piercing member directing device (108) changing its orientation as it progresses along grooves (104) and (106). As shown here, tissue-piercing member (116) adopts increasingly steeper orientations relative to device (100) as tissue-piercing member (116) advances. However, it should be understood that other variations of devices for forming tracts in tissue may comprise tissue-piercing members that advance along different trajectories during use.

FIG. 1G shows device (100) near the beginning of deployment of the tissue-piercing member. As shown there, tissue-piercing member (116) has a longitudinal axis (LA1), and the bottom surface (130) of housing (102) has a longitudinal axis (LA2). Longitudinal axes (LA1) and (LA2) have an angle (α₁) therebetween. In some variations, angle (α₁) as shown in FIG. 1G may be at least about 0° and/or at most about 30°. For example, angle (α₁) as shown in FIG. 1G may be from about 0.5° to about 30° (e.g., from about 0.5° to about 20°, from about 0.5° to about 10°, from about 0.5° to about 5°) or from about 0° to about 10° (e.g., from about 0° to about 5°).

Referring now to FIG. 1H, as tissue-piercing member (116) continues to advance, angle (α₁) increases (e.g., to a range of about 1° to about 45°, such as about 2° to about 30°). Similarly, FIGS. 1I and 1J depict additional increases in the size of angle (α₁) upon continued advancement of tissue-piercing member (116). As shown in FIG. 1I, for example, angle (α₁) may be from about 10° to about 60° (e.g., from about 10° to about 30°). Alternatively or additionally, as shown in FIG. 1J, angle (α₁) may be from about 12° to about 90° (e.g., from about 12° to about 60°, or from about 20° to about 90°).

The deployment angles that are used during a tissue tract-forming method may be selected, for example, based on the characteristics of the tissue. As an example, in some variations in which a device is being used to form a tract in relatively thin tissue (e.g., intestinal wall tissue), the device's tissue-piercing member deployment angles may be relatively small. In certain cases, the final tissue-piercing member deployment angle (e.g., the angle used to pass through the tissue and into a lumen) may be relatively large. For example, in cases in which the tissue may tent or may be pushed away by the advancing tissue-piercing member, it may be desirable to employ a relatively large deployment angle to be able to successfully pierce the tissue.

It should be understood that the different deployment angles shown in FIGS. 1G-1J are only exemplary, and other variations of devices may have other arrangements of deployment angles. For example, a device may have fewer different deployment angles, or may have a greater number of different deployment angles. Moreover, while FIGS. 1G-1I depict the angle between longitudinal axes (LA1) and (LA2) as increasing during deployment, in some cases such an angle may decrease during certain points in deployment. In certain variations, the tissue-piercing member directing device may be operated to achieve a specific angle of advancement of the tissue-piercing member. In some variations, the desired angle of advancement of the tissue-piercing member may become steeper as the tissue wall becomes thicker.

FIGS. 1K-1Q show device (100) being used to form a tract in a tissue wall (200). First, device (100) is delivered to the target site—here, tissue wall (200). Device (100) may be delivered to a target site using, for example, a NOTES procedure. As an example, if tissue wall (200) is a stomach wall, then device (100) may be delivered to tissue wall (200) via the esophagus. In such trans-esophageal applications, the device may have a size configured to fit within an esophagus having a diameter of about 13 millimeters, for example. Other NOTES approaches to target sites may be trans-gastric, trans-vaginal, or trans-rectal. Additionally, some variations of devices and/or methods may be used in hybrid NOTES procedures involving both NOTES techniques and laparoscopic techniques, or in other hybrid procedures.

Housing (102) of device (100) may comprise any appropriate material or combination of materials, and typically may comprise one or more biocompatible materials. In some variations, housing (102) or a portion thereof (e.g., its peripheral outer edge and/or tissue-contacting surfaces) may be made of one or more relatively soft materials (e.g., relatively soft plastics or polymers), while the inner components of device (100), and/or the more central regions of housing (102), may be more rigid. This may, for example, allow device (100) to be relatively easily maneuvered to a target site during a procedure, while still maintaining structural integrity and sufficient robustness.

Examples of materials which may be suitable for use in housing (102) include polymers, such as polyacetals (e.g., DELRIN® acetal resin), polystyrene, polyetheretherketone (PEEK), polyetherketoneketone (PEKK), polyethylene, acrylonitrile butadiene styrene (ABS), polyethylene terephthalate (PET), polycarbonates, polytetrafluoroethylene (e.g., TEFLON® polymer), polyimides, nylons, silicone, SANTOPRENE® thermoplastic vulcanizates, and polyvinyl chloride (PVC). Some types or families of polymers may be available in different durometers or hardnesses, and in such cases the appropriate polymer or polymers for the desired characteristics may be used. Examples of materials which may be relatively rigid include PEEK, PEKK, ABS, or silicone, and examples of materials which may be relatively soft include silicone, SANTOPRENE® thermoplastic vulcanizates, and PEBAX® polymers. Of course, these are only exemplary materials, and other relatively rigid or relatively soft materials may also be used, as appropriate. Additionally, materials that are not especially soft or rigid may be used. Moreover, in some variations, combinations (e.g., mixtures) of different materials may be used. For example, a blend of polymers may be used, or a composite of one or more polymers and filler materials (e.g., glass fibers and/or particles, carbon fibers, etc.) may be used.

In some variations, device (100) may be delivered to a target site laparoscopically, and may be scaled to fit within a trocar used in laparoscopic surgery. In certain variations, device (100) may be advanced within a sheath to a target site. Device (100) may have a smooth and/or rounded profile (e.g., cup- or clamshell-shaped) that may assist in its advancement through such a sheath. In some variations, device (100) may be delivered to a target site via one or more surgical incisions. For example, device (100) may be delivered directly to a vessel via an incision in the skin that is cut down to the vessel. In certain variations, device (100) may be incorporated into a larger overall device that may then be delivered to a target site, as discussed in additional detail below. While device (100) is discussed here, it should be understood that features and applications of device (100) may be applied to other devices described here, and vice-versa, as appropriate. Moreover, features of any methods described here may be applied to other methods described here, as appropriate.

As shown in FIG. 1K, first device (100) is aligned with a surface (202) of tissue wall (200). For example, if tissue wall (200) is a heart tissue wall, then surface (202) may be an exterior surface of the heart tissue wall. Device (100) may be aligned such that housing (102) is flush with surface (202), as shown, or alternatively may be aligned such that only a portion of housing (102) contacts surface (202), or even such that none of housing (102) contacts surface (202). Referring again to FIG. 1K, as shown, a portion of tissue wall (200) is drawn into indented portion (119) of housing (102). This may be achieved, for example, by contacting surface (202) against the surface of the tissue and moving device (100) in the direction of arrow (A8). Alternatively or additionally, in some variations, a device may comprise an indented portion having a suction or vacuum feature, or a portion that is not indented but that has a suction or vacuum feature, which may be used to draw tissue into a desired location. Moreover, while an indented portion has been described, in some variations, a portion having a different configuration may be used to draw tissue in, or to otherwise position or isolate tissue. For example, a device may comprise a more rounded groove portion that serves a similar function to indented portion (119).

As shown in FIG. 1L, after device (100) has been placed in the desired position, the portion of tensioning member (118) exiting housing (102) at second location (126) may be pulled in the direction of arrow (A1) to initiate advancement of tissue-piercing member (116) into tissue wall (200). The tissue-piercing member may then continue to be advanced through the tissue wall (FIG. 1M), until it has passed through the tissue wall (FIGS. 1N and 1O). For example, in cases in which tissue wall (200) is a vessel wall, the tissue-piercing member may be advanced through the vessel wall until the tissue-piercing member enters a lumen defined by the vessel wall. In this way, tissue-piercing member (116) forms a tract (204) in tissue wall (200) (FIG. 1Q). The features of tract (204) may be selected, for example, based on one or more characteristics of tissue wall (200).

In some cases, a tract such as tract (204) may be formed relatively easily using device (100), and/or using one or more of the other devices and/or methods described here. For example, by simply pulling on tensioning member (116) after properly positioning device (100), an operator may form a tissue tract. Of course, while a tensioning member has been described, other forms of controls may alternatively or additionally be employed. As an example, in some variations, one or more slide actuators, switches, buttons, or other similar features may be used to operate a tract-forming device. These actuating features may be proximally located, or may be otherwise situated in a location configured for ease of use by the operator.

Device (100) may be used for form a tract in a portion of tissue having a relatively consistent thickness, or in a portion of tissue having a variable thickness. Additionally, in some variations, a device may have a tissue-piercing member that can traverse two or more different types of tissue. For example, a device may have a tissue-piercing member with two different trajectories, one of which may be used to traverse the serosa of the stomach, and the other of which may be used to traverse the mucosa of the stomach.

While the method shown and described above is one way of forming a tract through tissue, other suitable methods may of course be used. For example, some methods may employ a combination of device-activated tissue-piercing member advancement and manual tissue-piercing member advancement. Additionally, in certain variations, a first portion of a tract may be formed by a first tissue-piercing member, and a second portion of the tract may be formed by a second, different tissue-piercing member. Any suitable number and combination of tissue-piercing members may be used to form a tissue tract.

In some variations, after a tissue-piercing member has formed a tract in a tissue wall, the tissue-piercing member may remain in the tissue wall while one or more guidewires and/or other tools or devices are advanced through or over the tissue-piercing member. In this way, the tissue-piercing member may provide the guidewires, tools, and/or devices with access and passage through or into the tissue wall. In certain variations, a guidewire may be passed through the tissue-piercing member, and the tissue-piercing member may be withdrawn. The guidewire may then be used to advance one or more tools or devices through the tissue wall. The tools that are advanced through a tract may be used, for example, in diagnostic and/or therapeutic procedures.

FIG. 1P depicts tissue-piercing member (116) being withdrawn from tissue wall (200) after tract (204) has been formed in tissue wall (200). As shown there, the portion of tensioning member (118) exiting housing (102) at first location (124) may be pulled proximally (in the direction of arrow (A6)) to withdraw the tissue-piercing member from the tissue wall. An operator may elect to withdraw a tissue-piercing member, for example, when the tissue-piercing member is no longer needed, or when it has inadvertently been deployed to a non-target site.

Referring now to FIG. 1Q, and as discussed above, tract (204) is formed by tissue-piercing member (116). As shown in FIG. 1Q, tract (204) has a gently sloping shape with a length (L) and a height (H). It should be understood, however, that tract (204) is only one example of a tract configuration that may be formed through tissue, and other appropriate configurations may alternatively or additionally be used. As an example, a tract may be more angular, or may be diagonal, or may have one or more diagonal portions. In some variations, a tract may comprise one or more sloped regions, one or more flat regions, and/or one or more regions that are substantially parallel to a longitudinal axis and/or surface of the tissue wall. In certain variations in which a tract is formed in a vessel wall (e.g., an artery wall), the tract may comprise one or more regions that are substantially parallel to a longitudinal axis of a lumen of the vessel and/or that are substantially parallel to a surface of the vessel wall. In some variations, a tissue-piercing member may be configured to advance into tissue along an undulating path, and may thereby form an undulating tract through the tissue. The undulating tract may, for example, have a greater surface area than tracts formed by other tissue-piercing members that follow a relatively straight path. This greater surface area may allow for the tract to self-seal relatively easily (e.g., for one or more reasons described below). The extent of undulation in a tract may in some cases be subtle or substantial. Other configurations of tracts (e.g., sawtooth tracts, oscillating tracts, etc.) may also be formed, as suitable for the particular application at hand.

The length of a tract (such as length (L) of tract (204)) may be any suitable or desirable length. In some variations, the length may be selected to help facilitate relatively rapid sealing of the tract. For example, when the devices and methods described here are used with the vasculature, a longer tract may be desirable, as it is believed that a longer tract may expose helpful biological factors (e.g., growth factors, tissue factors, etc.) that may aid in sealing the tract. This may also be the case with other tissue as well. In addition, a longer tract will have a larger area for mechanical pressure to act on, which may cause the tract to seal more quickly. In some variations, length (L) may be greater than the thickness of tissue wall (200) (e.g., in the location of tissue wall (200) where tract (204) is formed, or relative to the average thickness of tissue wall (200)). Here, of course, height (H) of tract (204) is equal to the thickness of tissue wall (200). However, in some cases, a tract may have a height that is shorter than the tissue wall thickness. For example, a tract may be formed to deposit one or more therapeutic agents into an interior section of a portion of tissue. In certain variations in which a tract is being formed in a vessel wall, a portion (e.g., a minority or a majority) of the tract may traverse the vessel wall substantially parallel to a longitudinal axis of the vessel wall and/or to a surface of the vessel wall.

Tracts may have any appropriate orientation or configuration relative to the tissue in which they are formed. As an example, FIG. 1R shows an exemplary tract (206) in a tissue wall (208). As shown there, tract (206) forms a diagonal path through tissue wall (208). Tissue wall (208) has an exterior surface (210) and an interior surface (212). For example, in some variations, tissue wall (208) may be a vessel wall, and interior surface (212) may face a lumen of the vessel wall. As shown in FIG. 1R, there is an angle (α₂) between tract (206) and interior surface (212). In some variations, angle (α₂) may be less than or equal to about 30° (e.g., less than or equal to about 19°, less than or equal to about 15°, less than or equal to about 10°, less than or equal to about 5°). In certain variations, angle (α₂) may be from about 1° to about 30° (e.g., from about 1° to about 19°, from about 1° to about 15°, from about 1° to about 10°, from about 1° to about 5°, from about 5° to about 15°, or from about 5° to about 10°). In some variations, angle (α₂) may be selected based on the tissue or procedure at hand.

FIG. 1S shows another tract (214) through a wall (216) of a vessel (218) having a lumen (220). As shown there, lumen (220) has a longitudinal axis (LA3), and tract (214) forms an angle (α₃) with longitudinal axis (LA3). In some variations, angle (α₃) may be less than or equal to about 30° (e.g., less than or equal to about 19°, less than or equal to about 15°, less than or equal to about 10°, less than or equal to about 5°). In certain variations, angle (α₂) may be from about 1° to about 30° (e.g., from about 1° to about 19°, from about 1° to about 15°, from about 1° to about 10°, from about 1° to about 5°, from about 5° to about 15°, or from about 5° to about 10°).

In certain variations, a tract through tissue may have multiple (i.e., at least two) different regions that are angled differently with respect to a reference, such as a surface of the tissue, a longitudinal axis of the tissue, or, in the case of a vessel, a longitudinal axis of a lumen of the vessel. As an example, FIG. 1T shows a tract (222) through a tissue wall (224) having an exterior surface (226) and an interior surface (228). Tract (222) comprises a first region (230), a second region (232) that is angled with respect to the first region, and a third region (234) that is angled with respect to the second region. In FIG. 1T, dashed lines (228′) and (228″), which are parallel to interior surface (228), are used to help illustrate the position of different regions of tract (222) relative to interior surface (228). As shown there, first region (230) forms an angle (α₄) with respect to dashed line (228″) (and, therefore, with respect to interior surface (228)), second region (232) forms a different angle (α₅) with respect to dashed line (228′) (and, therefore, with respect to interior surface (228)), and third region (234) forms yet another different angle (α₆) with respect to interior surface (228). In some variations, angle (α₄) may be from about 0.5° to about 45° (e.g., from about 1° to about 30°), angle (α₅) may be from about 1° to about 90° (e.g., from about 5° to about 60°), and/or angle (α₆) may be from about 0.5° to about 45° (e.g., from about 1° to about 45°). Of course, while each of these angles is different from the others, in some variations, at least two different regions of a tract through tissue may form the same angle with respect to a surface of the tissue and/or a longitudinal axis of the tissue. Moreover, and as discussed above, tracts need not have the configurations shown here, which are only exemplary. For example, a tract may have more or fewer regions than those shown here, may have a combination of curved regions, angular regions, and/or oscillating regions, or may have any other appropriate configuration.

Pressure, such as blood pressure, may act on a tract formed using one or more of the devices and/or methods described herein, and may thereby cause the tract to seal relatively rapidly without the need for an additional closure device. For example, the tract may seal in 15 minutes or less (e.g., 12 minutes or less, 10 minutes or less, 9 minutes or less, 6 minutes or less, 5 minutes or less, 3 minutes or less, 1 minute or less, etc.), reducing the duration of external compression, if any, that may be needed. Of course, if desirable, one or more additional closure devices (e.g., plugs, clips, glue, sutures, etc.) may be used, and/or additional closure methods (e.g., application of mechanical pressure, application of suction, application of one or more sealing agents, etc.) may be used. These additional closure devices and/or methods may help to expedite the sealing process.

As discussed above, in certain variations, a self-sealing tissue tract may be formed. A self-sealing tissue tract does not need interventional devices or methods to help it seal—by definition, it seals by itself. For example, a self-sealing tissue tract does not need a plug, energy, sealants, clips, sutures, or the like to help it seal. In some cases, the angle between the tissue-piercing member and the surface of the tissue or the longitudinal axis of the tissue wall (e.g., vessel wall) may be selected to form a self-sealing tract. For example, the angle may be relatively shallow, such as less than or equal to about 30° (e.g., less than or equal to about 19°, less than or equal to about 15°, less than or equal to about 10°, less than or equal to about 5°, from about 1° to about 30°, from about 1° to about 19°, from about 1° to about 15°, from about 1° to about 10°, from about 1° to about 5°, from about 5° to about 15°, or from about 5° to about 10°).

FIG. 1V shows an exemplary self-sealing tract (280) through a portion (282) of a wall (283) of a vessel (284), where the vessel has a lumen (286). As shown there, tract (280) is generally diagonal, and has a length (L2). The length of the tract may be any suitable or desirable length to help facilitate relatively rapid sealing of the tract. For example, when the devices and methods described here are used with the vasculature, a longer tract may be desirable. This is because it is believed that, as discussed briefly above, a longer tract may expose helpful biological factors (e.g., growth factors, etc.) that may aid in sealing the tract. This may also be the case with other tissue as well. In addition, a longer tract may have a relatively large area for mechanical pressure to act on, which may cause the tract to seal more quickly. In some variations, length (L2) may be greater than the thickness of portion (282) of wall (283) (e.g., in the location of portion (282) where tract (280) is formed).

The arrows shown in FIG. 1V illustrate how pressure acting on the tract may cause the tract to seal relatively rapidly, without the need for an additional closure device. The tract may seal when opposing tissue portions along the tract, such as opposing tissue portions (290) and (292), contact each other and form a seal. In some variations, the tract may seal in 15 minutes or less, 12 minutes or less, 10 minutes or less, 9 minutes or less, 6 minutes or less, 3 minutes or less, 1 minute or less, etc., reducing the duration of external compression, if any, that may be needed. Of course, if desirable, one or more additional closure devices (e.g., plugs, clips, glue, sutures, etc.) may be used.

Methods described here may include other aspects aside from just tract formation. For example, the methods may also comprise application of energy, delivery of one or more fluids or useful agents, delivery of one or more useful tools to a tissue site (e.g., through the tract), sensing temperature, pressure and/or blood flow, performing one or more procedures, visualization, determining the location of the device with respect to the tissue, combinations thereof, and the like. For example, while not shown, in some variations, tissue-piercing member (116) may be coupled to a tubular member that, in turn, is coupled to one or more ports (e.g., on the device or separate from the device). The tubular member may move in unison with the tissue-piercing member, and may cause the tissue-piercing member to be in fluid communication with the port or ports. In this way, one or more therapeutic agents may be delivered through the tissue-piercing member via the port or ports and the tubular member, and may eventually enter the tissue being treated.

In some variations, one or more of the devices described here may be rotated, repositioned, and/or otherwise manipulated. In certain variations, one or more of the devices described here may be configured for robotic and/or remote manipulation or operation.

While the formation of a single tract through tissue has been described, in some variations, multiple tracts may be formed through tissue. The tracts may be formed by the same device, or may be formed by multiple different devices. As an example, in some variations, a single device may comprise two different tissue-piercing members that may be deployed simultaneously or one after the other, in order to form two different tracts in tissue.

Tissue-piercing members for use with the devices and/or methods described herein may have any appropriate size, shape, and configuration. They may also comprise any appropriate material or materials, such as stainless steel or superelastic and/or shape-memory materials (e.g., Nitinol). In some variations, a tissue-piercing member may be hollow, while in other variations, a tissue-piercing member may be solid. Certain variations of tissue-piercing members may comprise one or more lumens and/or apertures. This may, for example, allow a wire to be advanced through the tissue-piercing member (and, e.g., into a vessel lumen, in cases in which the tissue comprises a vessel). In some variations, a device may comprise a tissue-piercing member in the form of a needle within a needle. Certain variations of devices may comprise more than one tissue-piercing member. As an example, a device may comprise a first tissue-piercing member having a relatively large cross-sectional diameter, and a second tissue-piercing member having a relatively small cross-sectional diameter. The relatively large tissue-piercing member may be selected, for example, to form a tract for deployment of a relatively large tool to a target site, while the relatively small tissue-piercing member may be selected, for example, to form a tract for deployment of a relatively small tool to a target site. Such selectivity may allow one access device to be used for many different procedures. In some variations in which a device comprises multiple tissue-piercing members, at least two of the tissue-piercing members may be coupled to a tissue-piercing member directing device, while in other variations, at least two of the tissue-piercing members may be coupled to different tissue-piercing member directing devices, and/or at least one of the tissue-piercing members may not be coupled to any tissue-piercing member directing devices. In certain variations, a device may comprise multiple tissue-piercing members in the form of microneedles. The multiple microneedles may be used, for example, in drug delivery applications. Other types of tissue-piercing members may also be used in drug delivery applications.

In some variations, a device for forming one or more tracts in tissue may be used to help place a guidewire at a target site. The guidewire may be at least partially disposed within the tissue-piercing member prior to, during, and/or after advancement of the tissue-piercing member through tissue to form a tract in the tissue. Alternatively, the guidewire may be positioned in the tract after the tract has been formed and the tissue-piercing member has been withdrawn.

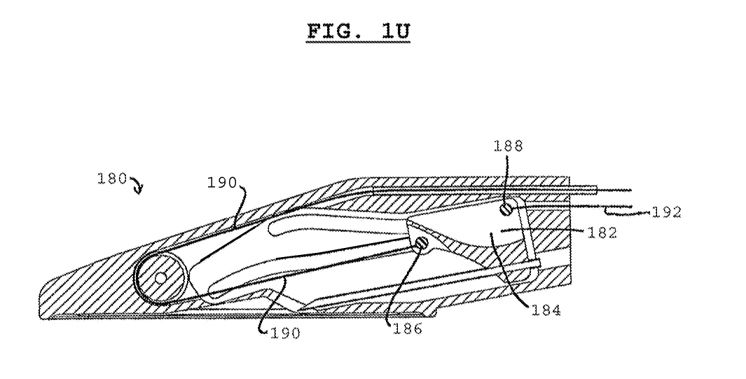

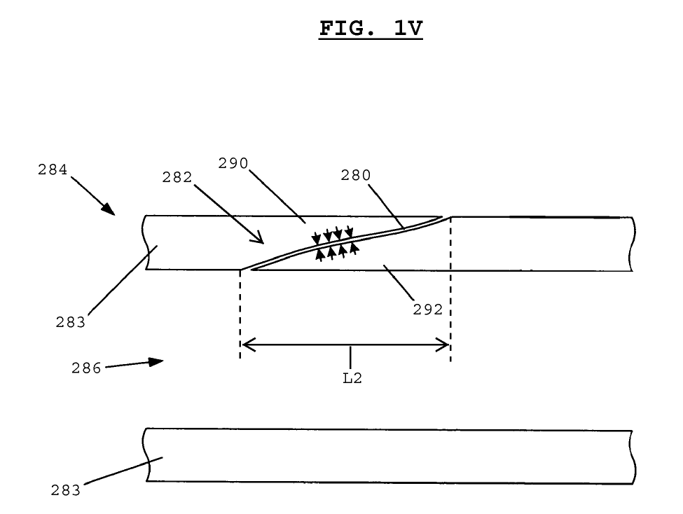

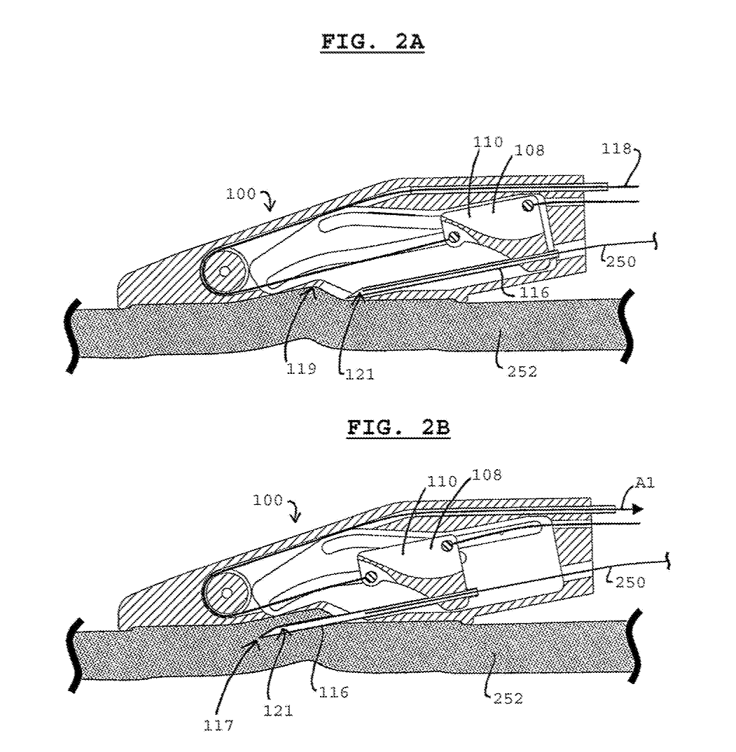

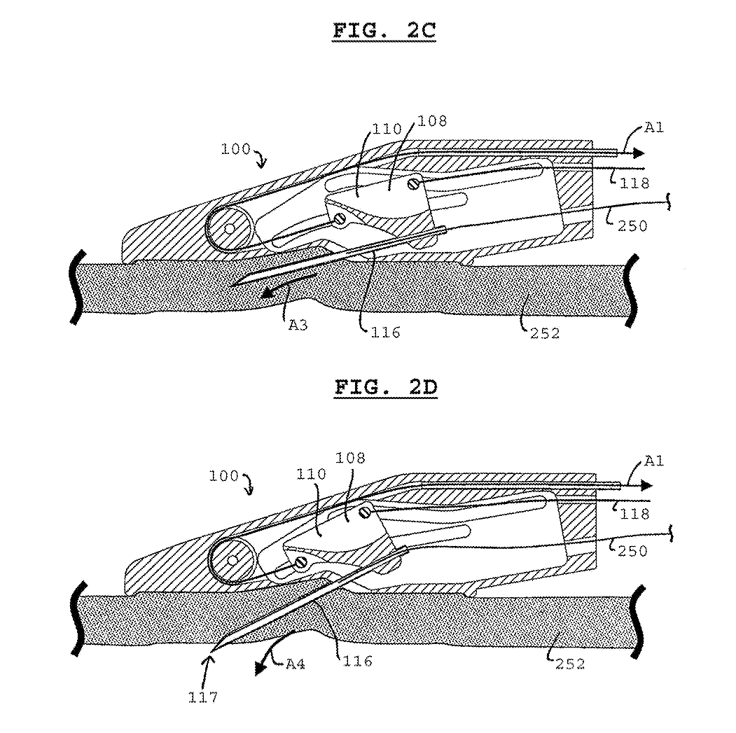

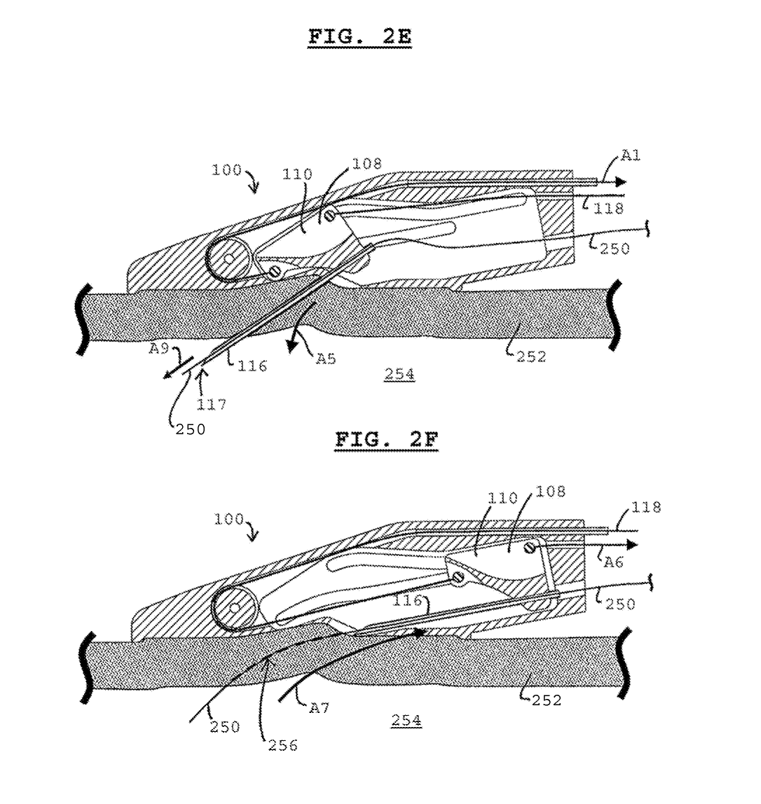

FIGS. 2A-2G show device (100) being used to place a guidewire (250) in a tissue wall (252). As shown in FIG. 2A, guidewire (250) is positioned within lumen (121) of tissue-piercing member (116), prior to advancement of the tissue-piercing member through the tissue wall. Device (100) may, for example, be sold or otherwise provided with guidewire (250) pre-positioned within lumen (121), or the operator or other medical personnel may load guidewire (250) into lumen (121) prior to use. In some variations, although not shown, the guidewire may be loaded into the tissue-piercing member after advancement of the tissue-piercing member has already begun, or even after the tissue-piercing member has already been used to form a tract in tissue.

Referring now to FIG. 2B, as tissue-piercing member (116) is advanced into tissue wall (252), guidewire (250) may not advance along with the tissue-piercing member. However, in some variations, the guidewire may be temporarily coupled to the tissue-piercing member, so that the guidewire moves in unison with the tissue-piercing member. As an alternative, in certain variations, an operator may manually advance the guidewire as the tissue-piercing member is being advanced.

As shown in FIGS. 2C and 2D, tissue-piercing member (116) continues to be advanced into tissue wall (252), until the tissue-piercing member has formed a tract in the tissue wall. Then, and referring now to FIG. 2E, guidewire (250) may be advanced through tissue-piercing member (116), in the direction of arrow (A9), so that guidewire (250) enters a space (254) on the other side of tissue wall (252). Thereafter, and as shown in FIG. 2F, tissue-piercing member (116) may be withdrawn from tissue wall (252), leaving guidewire (250) in place. The result, shown in FIG. 2G, is guidewire (250) positioned within a tract (256) formed by tissue-piercing member (116). It should be noted that while FIGS. 2A-2G depict the placement of a guidewire through tissue, other variations of components or devices may also be positioned using the same method or a similar method, as appropriate.

In certain variations, one or more surfaces of a device for forming one or more tracts in tissue may be configured to help grasp or otherwise secure the tissue during tract formation. For example, a device may have at least one textured surface, grooved surface, serrated surface, porous surface, spiked surface, abrasive surface, etc. In some variations, a device may include one or more features (e.g., ridges, hooks, barbs, etc.) that may be used to engage tissue.

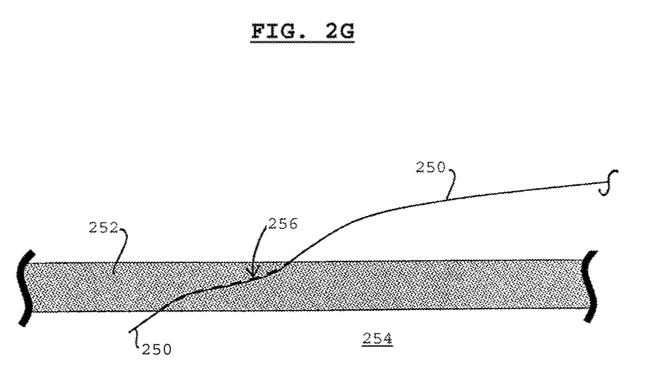

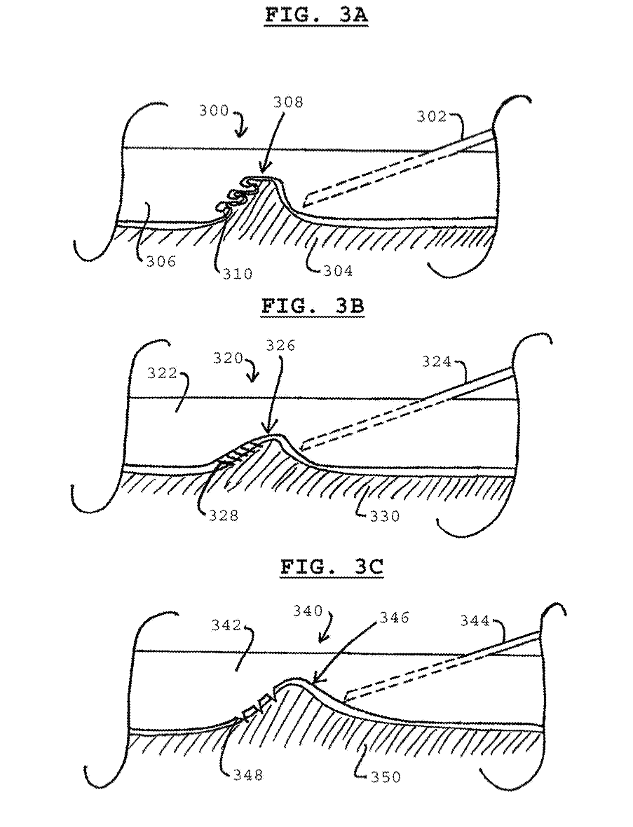

As an example, FIG. 3A shows a device (300) that may be used to help advance a tissue-piercing member (302) through tissue (304). As shown there, device (300) comprises a housing (306) having an indented portion (308) comprising fingers (310). Fingers (310) may help to temporarily couple device (300) to tissue (304), as shown in FIG. 3A. This temporary coupling may, for example, stabilize tissue (304) as tissue-piercing member (302) is advanced into and through tissue (304) to form a tract therein. As another example, FIG. 3B shows a device (320) comprising a housing (322), a tissue-piercing member (324) that may be advanced through the housing and into tissue, and an indented portion (326) comprising barbs or hooks (328). Barbs or hooks (328) may be used to engage tissue (as shown, tissue (330)), thereby helping position the tissue for piercing by tissue-piercing member (324). As an additional example, FIG. 3C shows a device (340) comprising a housing (342), a tissue piercing member (344) that may be advanced through the housing and into tissue, and an indented portion (346) comprising spikes (348). Similarly to fingers (310) and barbs or hooks (328), spikes (348) may be used to engage tissue (as shown, tissue (350)) and to thereby position or isolate the tissue for piercing by tissue-piercing member (344). Of course, these are only exemplary features, and any other appropriate features for engaging, isolating, and/or positioning tissue may be used. For example, in some variations, a device may comprise a housing having an indented portion including a vacuum port. A vacuum may be applied to draw tissue into the indented portion.

Additionally, it should be noted that in some variations, the engagement of tissue by such features may help to stabilize the device with respect to the tissue. Moreover, combinations of different types of surfaces and/or features may be used in some variations. For example, a device for forming one or more tracts in tissue may have a portion with a grooved surface and a portion with a spiked surface, or a portion with a serrated surface and a portion with a smooth surface, etc. Such surfaces and/or features need not be limited to an indented portion of a device; rather, they may be employed in any suitable location on a device, and in some cases may be employed in a device that does not include any indented portions.

In certain variations, a device for forming one or more tracts in tissue may include at least one surface comprising one or more coatings, such as a polymer coating. The coating or coatings may, for example, provide enhanced gripping of a tissue surface. As an example, in some variations, a device may comprise a surface with a silicone coating. In certain variations, a device may comprise a surface with one or more hydrophilic coatings and/or one or more hydrophobic coatings. As an example, one portion of a device may be coated with a hydrophilic coating, while another portion of the device is coated with a hydrophobic coating. Some variations of coatings may be porous coatings, and/or may comprise fibers, weaves, and/or other absorbent materials. Some such variations of coatings may aid in the removal or extraction of moisture or mucus (e.g., thereby increasing traction with the coupled or mated tissue surface). In certain variations, the type of coating that is used on at least a portion of a device for forming one or more tracts in tissue may be selected based on the type of tissue involved.

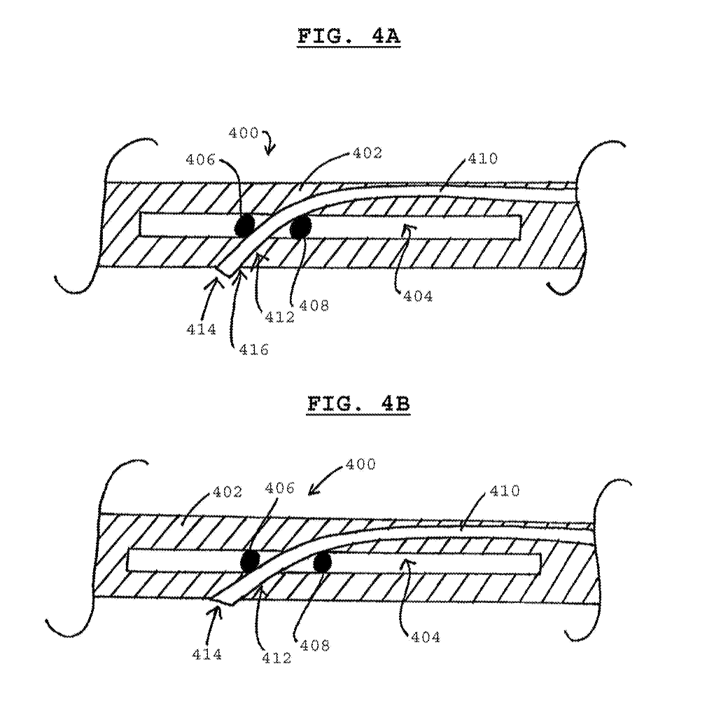

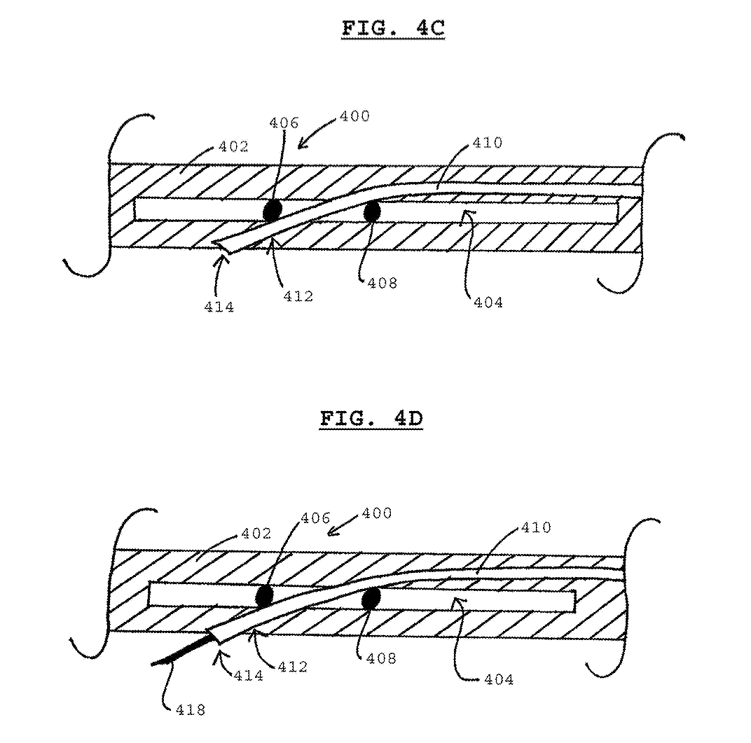

While one variation of a tissue-piercing member directing device has been described above, other variations of tissue-piercing member directing devices having other configurations may also be used to form one or more tracts in tissue. As an example, in some variations, a device may include at least one tubular member that may be manipulated to help position, direct, and/or advance a tissue-piercing member. For example, FIGS. 4A-4D show a tissue-piercing member directing device (400) comprising a housing (402) and a track (404) formed within the housing. Track (404) may, for example, be in the form of a groove or slot within the housing. Two protruding members (406) and (408) are disposed within the track and are capable of being slidably moved within the track.

Device (400) further comprises a tubular member (410) having a distal portion (412) that is positioned between protruding members (406) and (408). Tubular member (410) may be formed of, for example, one or more elastomeric materials. In some variations, tubular member (410) may be formed of one or more relatively flexible materials, such as silicone, stainless steel, cobalt chromium, Nitinol, or any other flexible metal alloy or polymer. The distal end (414) of tubular member (410) is coupled to housing (402) at a pivoting point (416) (FIG. 4A) about which the tubular member may pivot. For example, distal end (414) may be coupled to housing (402) by one or more pins, and/or may be disposed between one or more pins that are coupled to housing (402). In some variations, distal end (414) itself may comprise pins or tabs that are rotatable within corresponding recesses in housing (402). In certain variations, the recesses may be covered (e.g., with a thin coating material) to capture the pivoting pins or tabs. The pins or tabs may, for example, be welded, mounted, press-fit and/or molded onto tubular member (410). The presence of pivoting point (416) may allow distal end (414) of tubular member (410) to remain in the same general location relative to a tissue wall, regardless of the angle of tubular member (410) relative to the bottom surface of housing (402).

As shown in FIGS. 4A-4C, one or both of protruding members (406) and (408) may be moved into different positions to change the orientation of distal end (414) of tubular member (410). The orientation of distal end (414) may change because as the protruding members are moved into different positions, distal end (414) may rotate or pivot about pivoting point (416). When the desired position and orientation of the distal end have been achieved, a tissue-piercing member (418) (FIG. 4D) may be advanced through tubular member (410) (and, e.g., into a tissue wall). In certain cases, movement by tubular member (410) itself may cause one or both of protruding members (406) and (408) to move. For example, the protruding members may become more separated from each other as the angle of the tubular member with respect to the bottom surface of housing (402) becomes more shallow.

Tubular members for use with a tissue-piercing member directing device may be flexible, deformable, hinged and/or articulated, etc., such that they are capable of being relatively easily positioned for tissue-piercing member deployment. In some variations, a tubular member and/or a tissue-piercing member advanced therethrough may be relatively springy. This may help the tubular member to change positions relatively easily (e.g., using just one protruding member for positioning). In certain variations, a tubular member of a tissue-piercing member directing device may have a tapered wall section that allows the tip of the tubular member, and/or any other feature, to be preferentially more flexible relative to the rest of the tubular member. This may, for example, make it relatively easy to deform the tubular member in a certain region. In some variations, a device may include at least one tissue-piercing member positioning component that is not in the form of a tubular member. For example, a device may include a tissue-piercing member positioning component that is in the form of an elongated coil, where the tissue-piercing member is configured to pass through the center of the coil.

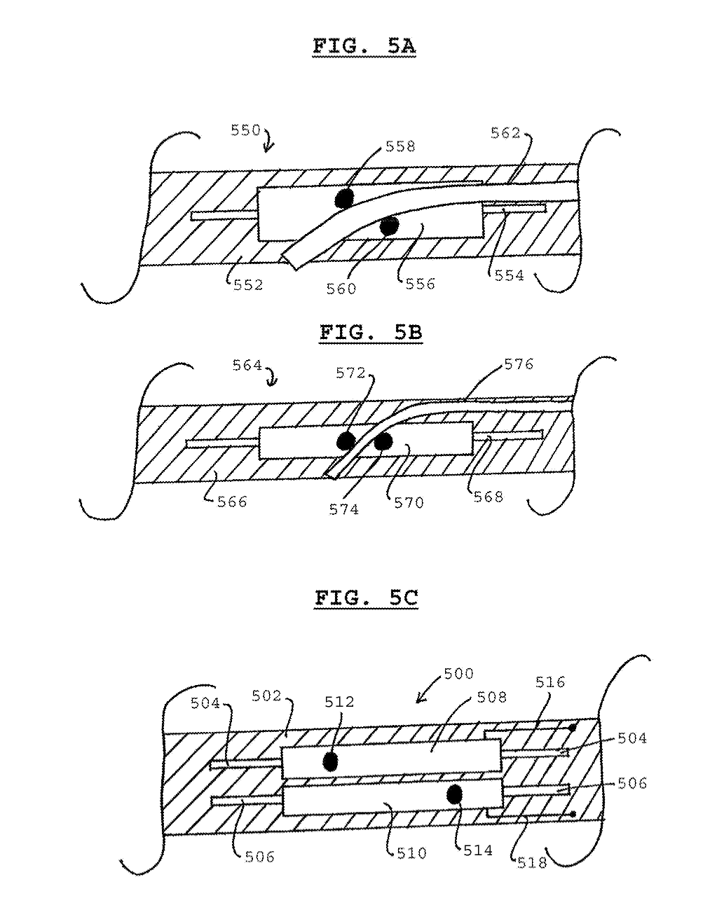

In certain variations, a device may comprise one or more protruding members that are disposed on one or more slidable members. The slidable member or members may, in turn, be slidably engaged with one or more tracks of a housing of the device. For example, FIG. 5A shows a device (550) comprising a housing (552) having a track (554) formed therein, and a slidable member (556) slidably engaged with track (554). Two protruding members (558) and (560) protrude from slidable member (556) and may be used to help position and orient a tubular member (562). As shown in FIG. 5A, protruding members (558) and (560) are offset with respect to each other. However, other variations of devices may alternatively or additionally include aligned protruding members. As an example, FIG. 5B shows a device (564) comprising a housing (566), a track (568) within housing (566), a slidable member (570) slidably disposed within track (568) and having two protruding members (572) and (574) protruding therefrom, and a tubular member (576) disposed between the two protruding members. As shown in FIG. 5B, protruding members (572) and (574) are aligned along slidable member (570). In some cases, adjustments to tubular members described herein may result in a passive separation or other movement of the protruding members. For example, as the curvature of a tubular member is decreased and/or as the angle of the tubular member relative to a bottom surface of the housing decreases (i.e., becomes more shallow), protruding members on either side of the tubular member may adjust to accommodate the change.

The members that are used to help position and orient a tubular member need not be disposed within one track, and may have different configurations. They may be manipulated in unison or in some cases, they may be capable of being manipulated independently of each other. For example, FIG. 5C shows a variation of a tissue-piercing member directing device (500) comprising a housing (502) and two tracks (504) and (506) formed within the housing. A slidable member (508) or (510) is disposed within each track, and each slidable member comprises a protruding member (512) or (514) protruding therefrom. Using manipulation wires (516) and (518), the slidable members (508) and (510) may be independently moved along tracks (504) and (506). While not shown, device (500) may comprise at least one tubular member disposed between protruding members (512) and (514). As described above, the tubular member may be used to position and orient a tissue-piercing member for advancement into tissue along a desired path.

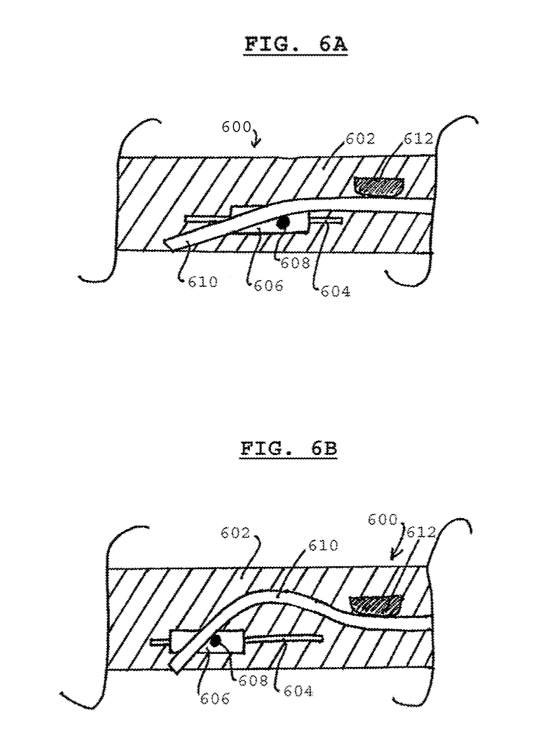

Still other variations of tissue-piercing member directing devices may be used. For example, FIGS. 6A and 6B show a tissue-piercing member directing device (600) comprising a housing (602) having a track (604) formed therein and a slidable member (606) slidably disposed within the track and comprising a protruding member (608). Device (600) also comprises a tubular member (610) and a boss (612) that may be used to position or displace the tubular member. For example, during use, boss (612) and/or slidable member (606) may be moved and adjusted until the desired position and orientation for tubular member (610) have been achieved. As an example, one or both of these features may be adjusted to move tubular member (610) from the position shown in FIG. 6A to the position shown in FIG. 6B. Alternatively, in certain variations, boss (612) may not be movable but may provide a location against which a portion of the tubular member may be positioned and/or stabilized. For example, in some variations, boss (612) may provide a reference point for initiating deflection of tubular member (610). Tubular member (610) may be coupled to protruding member (608) and/or boss (612), or may not be coupled to either of these features (e.g., the tubular member may simply be positioned between the two features). Moreover, in some variations, a device may include a boss and at least two slidable members that may be used together to position and orient a tubular member of the device.

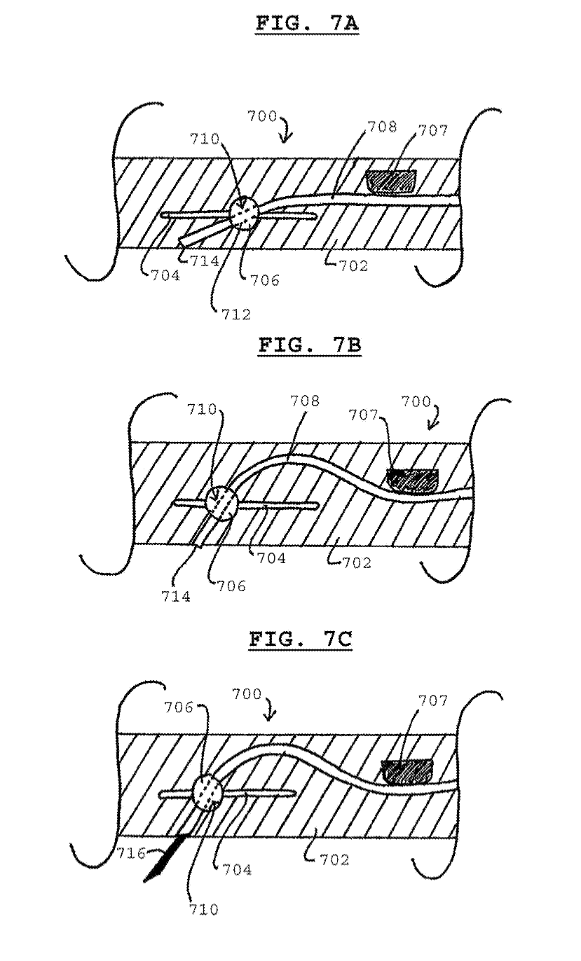

Additional configurations of tissue-piercing member directing devices may be used in a tissue tract formation procedure. For example, FIGS. 7A-7C depict a tissue-piercing member directing device (700) comprising a housing (702) having a track (704) formed therein, a rotatable member (706) disposed within the track (704), a boss (707) coupled to or integral with housing (702), and a tubular member (708). Tubular member (708) is routed beneath boss (707), and a distal portion (710) of tubular member (708) passes through a channel (712) within rotatable member (706). In addition to being capable of rotation, rotatable member (706) is slidably disposed in track (704). Thus, the boss and/or rotatable member may be manipulated to redirect the distal end (714) of tubular member (708) as demonstrated, for example, by FIGS. 7A and 7B. Referring now to FIG. 7C, a tissue-piercing member (716) may then be advanced through tubular member (708) (and, e.g., into a tissue wall).

Tubular member (708) may be coupled to rotatable member (706) and/or boss (707), or may not be coupled to either feature. Additionally, in some variations, a tubular member may be coupled to a device housing. As an example, tubular member (708) may be coupled to housing (702) of device (700). For example, a more proximal portion of the tubular member may be coupled to the device housing (thereby providing for easier manipulation of the distal portion of the tubular member), and/or the distal end of the tubular member may be coupled to the device housing at a pivoting point.

While rotatable members, bosses, protruding members, and slidable members have been described, any combination of any of these features may be used in a tissue-piercing member directing device. At least one of each such feature may be used, or in some variations, only certain features may be used, while other features may not be included in a device. A device may include only one such feature, or may include multiple such features (e.g., of the same type or different types). Moreover, other features suitable for positioning a tubular member and/or tissue-piercing member may be used, either as an alternative to, or in addition to, using one or more of the features described above.

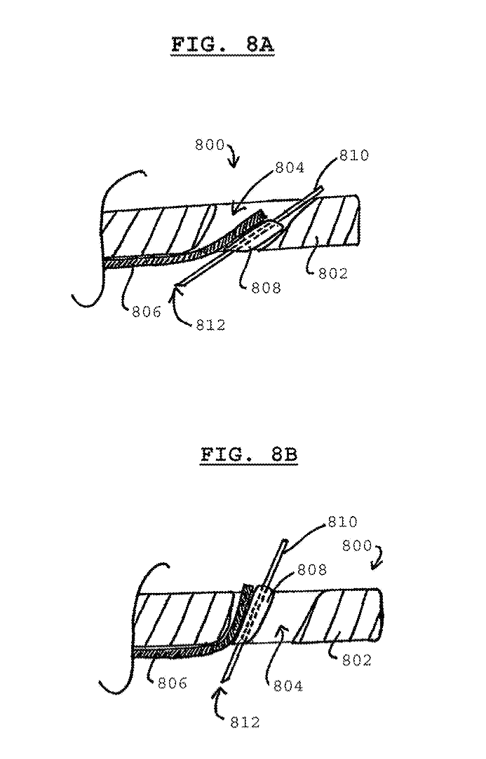

An additional example of a feature that may be used to direct a tissue-piercing member is a flexible member. As an example, FIGS. 8A and 8B show a device (800) for forming one or more tracts in tissue, where the device comprises a housing (802) having a groove (804) therein, and a flexible member (806) partially disposed within the groove. Flexible member (806) is coupled to a holder (808) through which a tissue-piercing member (810) is disposed. However, in some variations, a flexible member may be directly coupled to a tissue-piercing member. During use, flexible member (806) may be manipulated (e.g., as shown in FIG. 8B) to reposition holder (808). As a result, tissue-piercing member (810) is repositioned and the location of its tissue-piercing tip (812) changes. As shown, here tissue-piercing member (810) is both translated and rotated with respect to housing (802). This may allow for the point of rotation to be outside of device (800), and to be capable of shifting during pivoting. As a result, tissue deflection during advancement of the tissue-piercing member may be minimized. Device (800) is just one configuration of a device with a flexible member, and other configurations may alternatively be employed. As an example, in some variations, a device comprising multiple flexible members may be employed. Moreover, while a flexible member has been described, some variations of devices may alternatively or additionally comprise other types of flexing or moving mechanisms. For example, a device may include one or more hinges or articulating members that are coupled to a needle port, and that are capable of adjusting the position of the needle port.

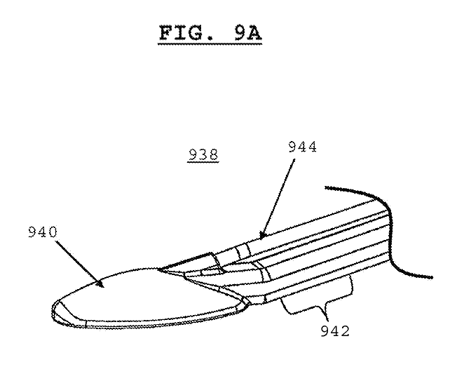

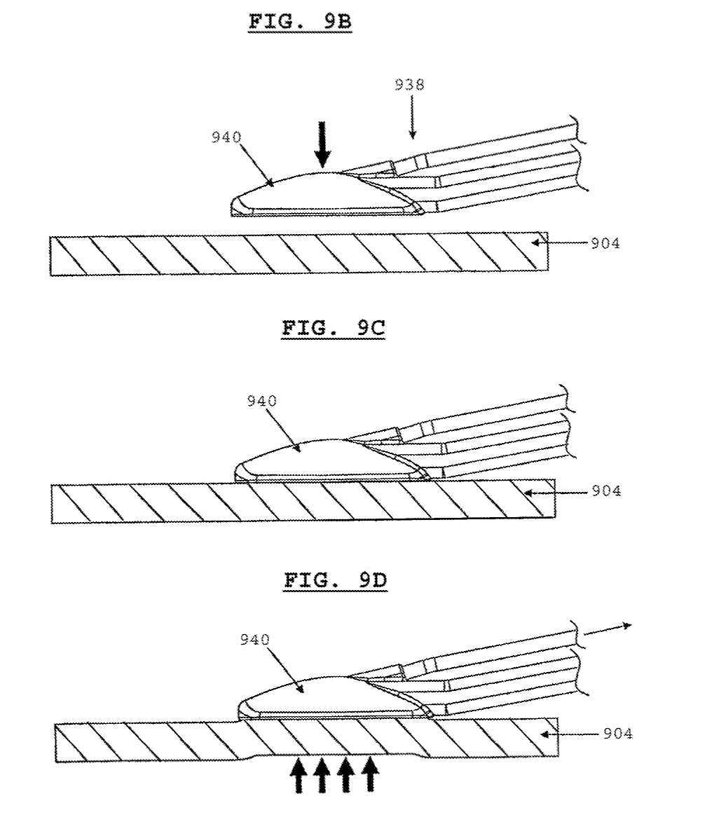

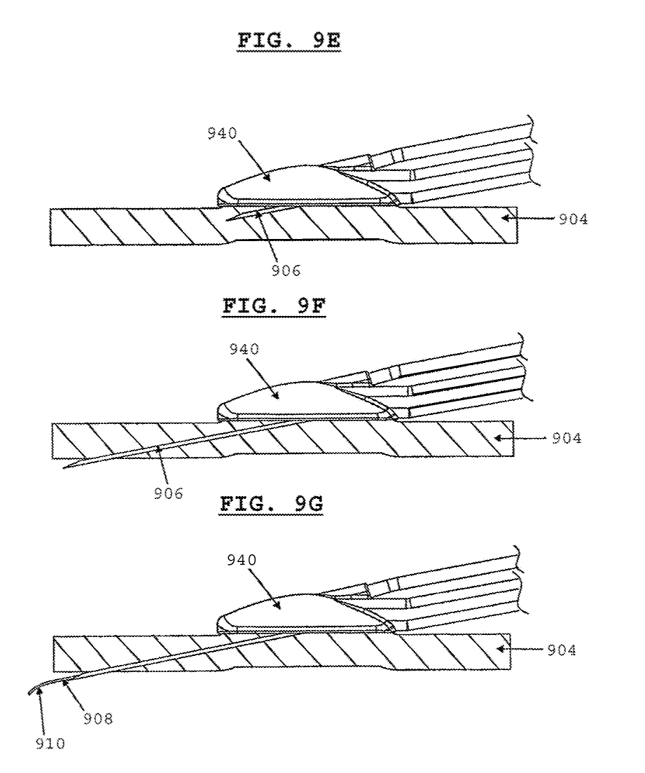

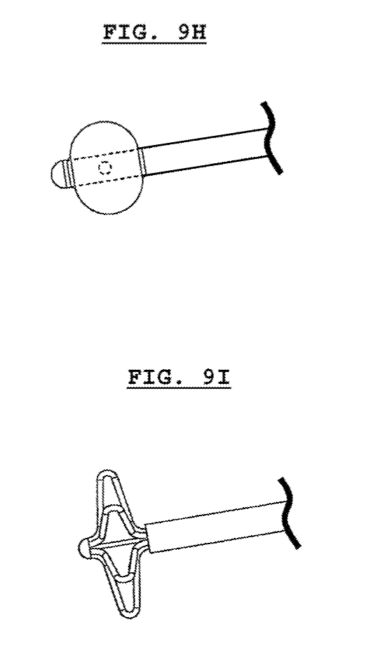

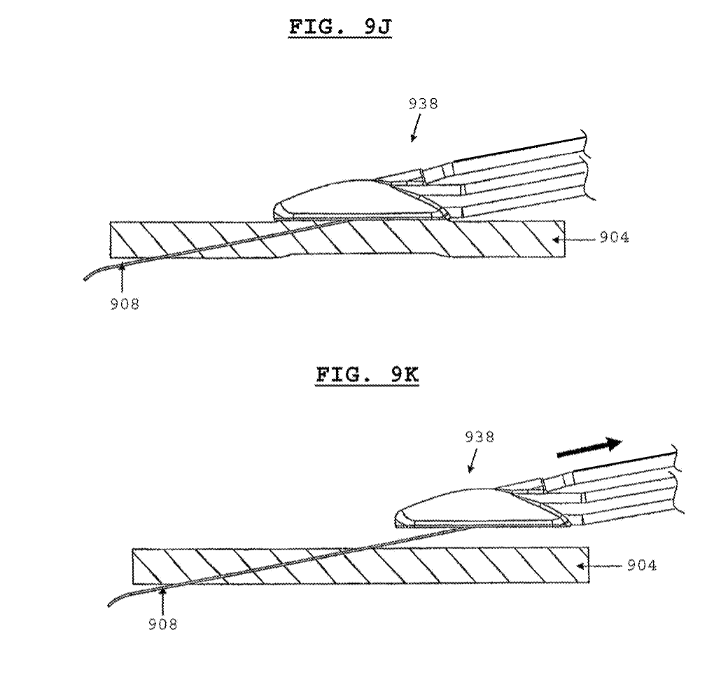

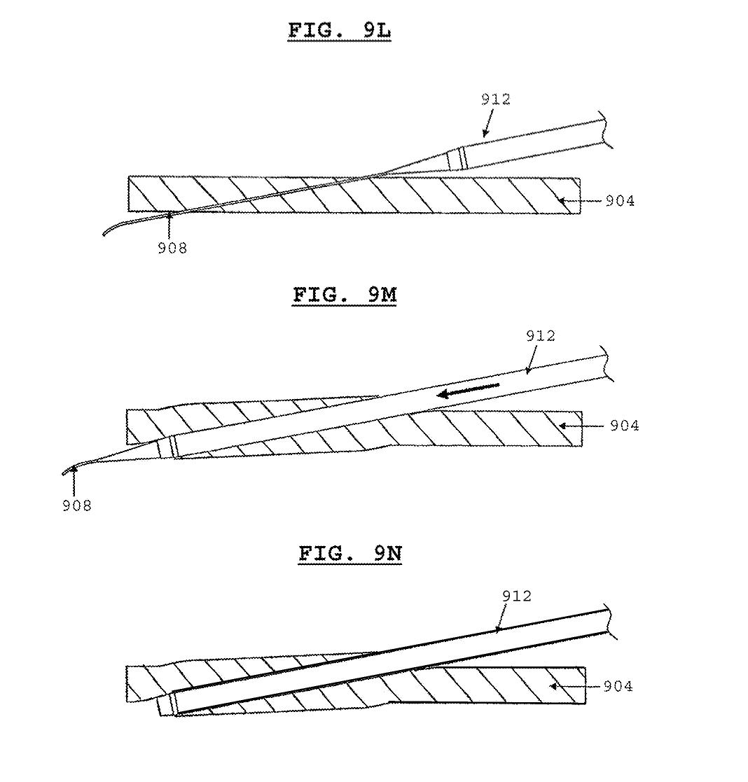

Tissue-piercing member directing devices such as those described above may be used on their own, or may be incorporated into another type of device. For example, FIG. 9A provides an illustration of a device (938) that may be used to position and isolate tissue for tract formation by a tissue-piercing member. In some variations, a tissue-piercing member directing device (not shown) may be incorporated into device (938). Device (938) may apply suction to tissue to draw the tissue into a certain position. This tissue drawing may be especially helpful, for example, during gastric procedures where the tissue can be difficult to manage. Then, a tissue-piercing member in the device may be advanced through the tissue to form one or more tracts in the tissue. In some variations, by applying suction and positioning the tissue, the device may prevent the tissue-piercing member from skimming the surface of the tissue. As shown in FIG. 9A, device (938) comprises a suction member (940), an elongate member (942), and a vacuum hose or connector (944). In use, suction member (940) may help to position or isolate tissue, as described in additional detail below.

In some variations, a device having one or more suction members may be advanced adjacent to tissue, suction may be applied to draw the tissue against the one or more suction members, and then a tissue-piercing member may be advanced through the drawn tissue to form a tract in (e.g., through) the tissue. During advancement, the tissue-piercing member may follow a path predetermined by a tissue-piercing member directing device. While FIG. 9A depicts a certain device (938), in some variations a device may comprise multiple suction members, one or more energy applicators (e.g., ultrasound, RF, light, magnetic, combinations thereof, etc.), one or more sensors (e.g., to sense temperature, pressure, blood flow, combinations thereof, etc.), more than one tissue-piercing member, more than one tissue-piercing member directing device, etc.

With specific reference now to the figures, FIGS. 9B-9N depict one illustrative method for forming a tract in tissue. As shown in FIG. 9B, device (938) comprising suction member (940) is advanced adjacent to tissue (904). Suction may then be applied to suction member (940) so that the suction member is pulled toward the tissue until it contacts the tissue, as shown in FIG. 9C. The tissue may also rise or deform up against or into the suction member, and/or within any features or openings in the tissue-contacting areas of the device, as shown by the arrows in FIG. 9D. Of course, suction may be applied at any stage of a tissue tract-forming method. For example, suction may always remain on, and the device may be advanced while suction remains on. Conversely, the device may be advanced adjacent to tissue and then suction may be applied, as shown here. Alternatively, suction may be toggled on and off, regulated, or otherwise modulated, to control the vacuum strength or flow.

Returning to the figures, once the tissue has been drawn against the suction member, the tissue-piercing member directing device (not shown) may be used to advance a tissue-piercing member through the drawn tissue to form a tract in the tissue (FIG. 9E). The tract may be of any length, and may traverse through the tissue, as shown in FIG. 9F. Once a tract has been formed, one or more tools may be advanced through the tract. For example, and as shown in FIG. 9G, a guidewire (908) may be advanced through the tissue-piercing member, and through the tract.

Guidewire (908) may be any guidewire having a diameter suitable for use with the corresponding tissue-piercing member (906). Guidewire (908) may also have one or more expandable members (e.g., an expandable balloon, such as shown in FIG. 9H; an expandable cage or flower wire formation, such as shown in FIG. 9I; expandable arms; etc.) or similar such features on its distal end (910). In this way, the distal end of the guidewire may be used to help locate or position the device with respect to the tissue and to maintain its position for a portion of the procedure. For example, guidewire (908) may be advanced through tissue (904), and the distal expandable feature expanded. Guidewire (908) may then be gently pulled proximally (i.e., in the direction of the tissue). Once the expandable member abuts the tissue (as determined, for example, via tactile feedback), the location of tissue has been determined and this information may be used as a guide for the rest of the procedure. Additionally, the expandable member may prevent accidental removal of the guidewire during a device exchange or removal and/or during advancement of a different device along the guidewire. Of course, the above-described tissue location methods may not be necessary when indirect (e.g., fluoroscopic guidance, ultrasound, etc.) or direct (e.g., camera, scope, etc.) visualization is employed, which visualization techniques may be used with any of the methods described here. Vacuum checks may also be useful in determining the location of the tissue, or the device with respect to tissue.

Turning back now to FIG. 9J, after guidewire (908) has been advanced through tissue (via a lumen in the tissue-piercing member for example), tissue-piercing member (906) may be withdrawn. Suction may be turned off, if desired, and the device may be withdrawn proximally, as shown in FIG. 9K. One or more dilators (or a single step-up dilator) or introducers (912) (FIG. 9L) may then be advanced over guidewire (908) if necessary to expand the tissue tract. Once sufficient access to the target site has been obtained, guidewire (908) may be withdrawn, as shown in FIGS. 9L-9N. One or more additional tools may then be introduced through the introducer, to carry out any suitable procedure. In some variations, the method described here may be used to carry out an arteriotomy to provide access to the vasculature. Once all procedures have been performed, the tools and introducer may be removed, allowing the tract to self-seal. Of course, as described above, sealing of the tract may be facilitated or expedited by mechanical pressure, delivery of energy (RF, ultrasound, microwave, etc.), and/or the use of one or more agents and/or closure devices, a combination of the foregoing, or the like.

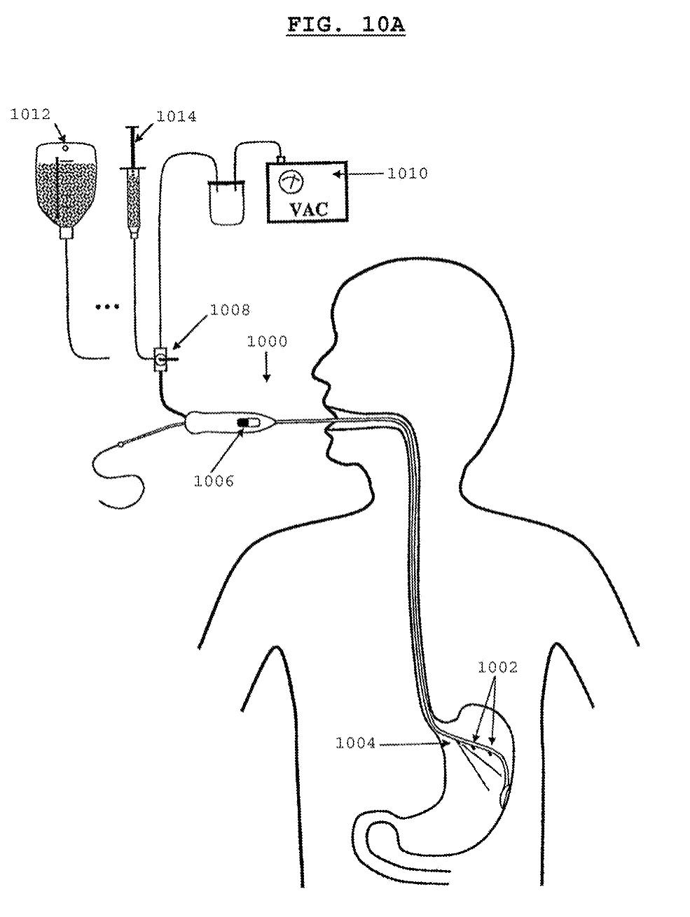

FIG. 10A is an overview illustration of how a suction device (1000) may be used to form one or more tracts in tissue within or through the stomach, or stomach tissue. In this variation, the device is not used with a separate gastroscope, and here visualization is enabled by a series of cameras or other visualization devices (1002) in combination with a light or illumination source (1004). This particular method may be quite useful, for example, in natural orifice transluminal endoscopic surgeries. FIG. 10A also details an illustrative proximal control of device (1000), here in the form of a slide actuator (1006). Slide actuator (1006) may be used, for example, to advance and retract the tissue-piercing member, to turn on and off one or more visualization devices (1002), to turn on and off illumination source (1004), or some combination thereof. In a device that includes a slide actuator as well as a tissue-piercing member directing device, the slide actuator may be capable of advancing and/or retracting the tissue-piercing member, and/or may be used to operate the tissue-piercing member directing device. As an example, in certain variations, a slide actuator may be used to actuate both the tissue-piercing member and the tissue-piercing member directing device in a controlled sequence to achieve the desired tissue-piercing path.

Some variations of devices may include multiple slide actuators. For example, a device may comprise a slide actuator that can be used to actuate a tissue-piercing member of the device, as well as a separate slide actuator that can be used to actuate a tissue-piercing member directing device of the device. Of course, while slide actuators have been described, a device may include any number and type of proximal controls (slides, switches, buttons, etc.) to control any number or combination of functions (e.g., vacuum, visualization, actuation of the tissue-piercing member and/or tissue-piercing member directing device, illumination, fluid flush, etc.).

Also shown in FIG. 10A is the illustrative use of suction, fluid injection, and the like. Here, a three-way valve (1008) is shown, which connects to and helps control use of vacuum (1010), bag infuser (1012), and syringe injector (1014). That is, three-way valve (1008) may be toggled between its various positions to turn off or on the vacuum, or fluid (via bag infuser or syringe injection). Having the ability to turn on and off the vacuum, for example, may be particularly useful in instances where the device has become stuck on or against one or more tissue surfaces. Turning on and off fluid injection or delivery, for example, may be particularly useful when it is desirable to flush, irrigate, unclog, or deliver one or more substances to the tissue. Of course, the control depicted in FIG. 10A is just one way to control or operate the described functions. It should be understood that any suitable configuration (having a two-way valve to control certain features, but not others, having additional proximal controls, combinations of the foregoing, and the like) may be used.

FIGS. 10B and 10C schematically represent variations where one or more fluids (therapeutic, flushing, sterilizing, etc.) are delivered to the tissue (1024) while the suction member is still under vacuum. For example, FIG. 10B depicts a suction member (1020) having one or more peripheral ports (1022) thereon or therealong for delivery or passage of one or more fluids therethrough (shown by arrows (1023)). In this variation, fluids may be injected or delivered through the one or more peripheral (1022) and/or other ports (e.g., needle ports, etc.), and then collected through a vacuum port (1026) while tissue (1024) remains captured by suction. FIG. 10C depicts an alternative variation where the fluid is not collected through a vacuum port (1036). Shown there is a suction member (1030) having one or more peripheral ports (1032) thereon or therealong for delivering one or more fluids (e.g., therapeutic, flushing, sterilizing, etc.) to tissue (1038). In this variation, the fluid is injected through a first syringe (1040) or other delivery system, and is collected by a separate second syringe (1042) or other suitable collection system, so that vacuum port (1036) need not function to collect fluid. The push-pull syringe of this variation may, for example, help prevent the vacuum from emptying syringe contents.

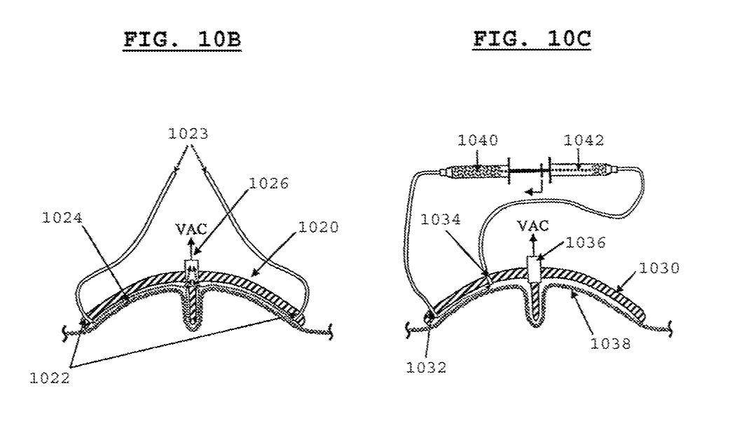

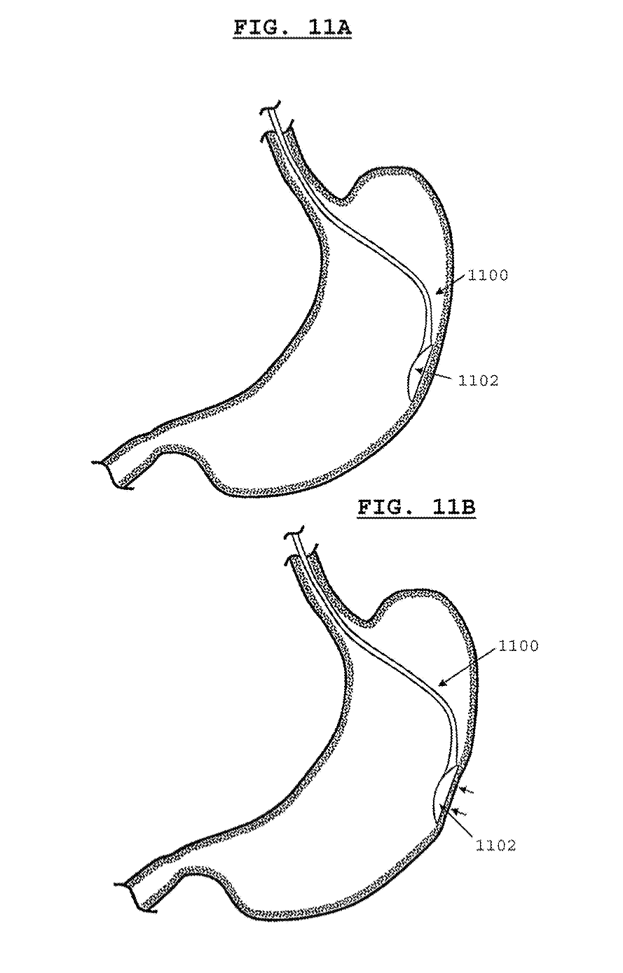

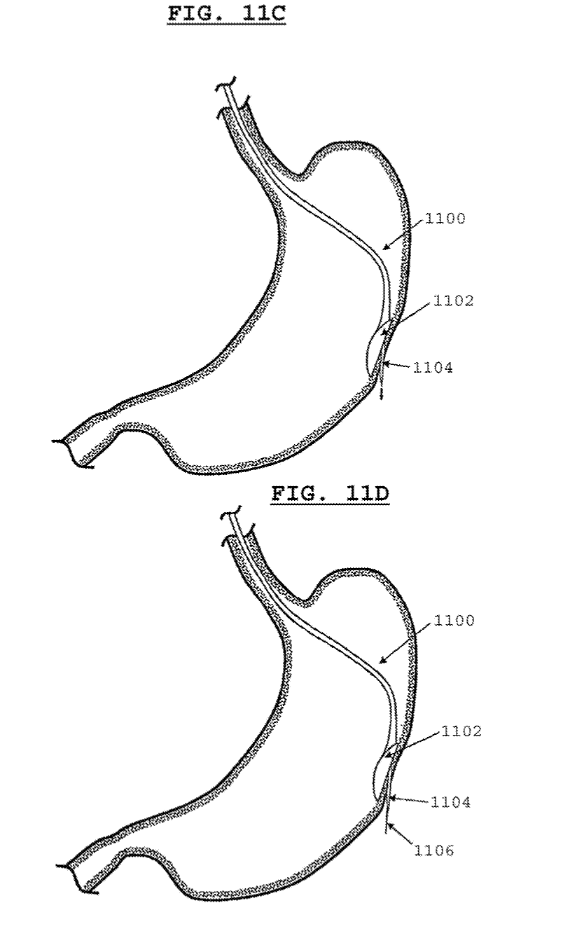

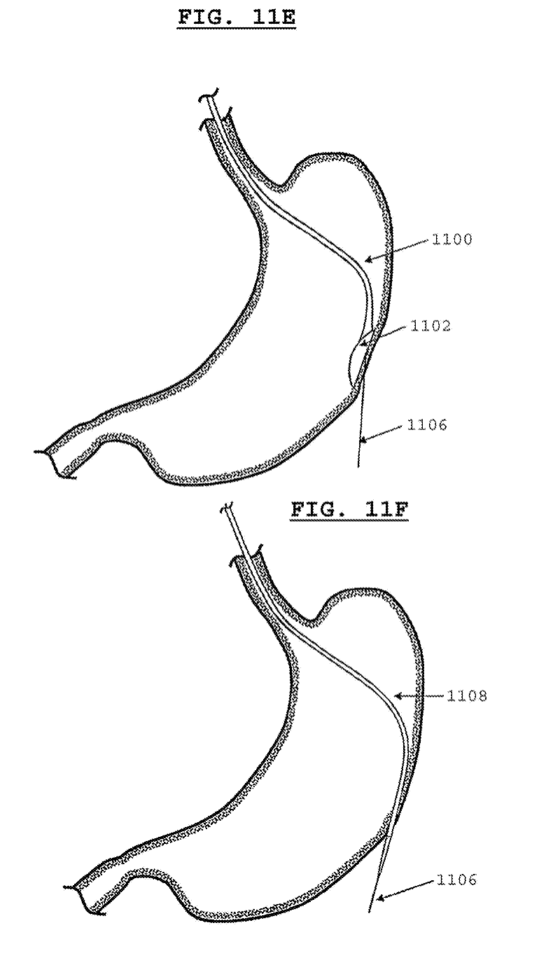

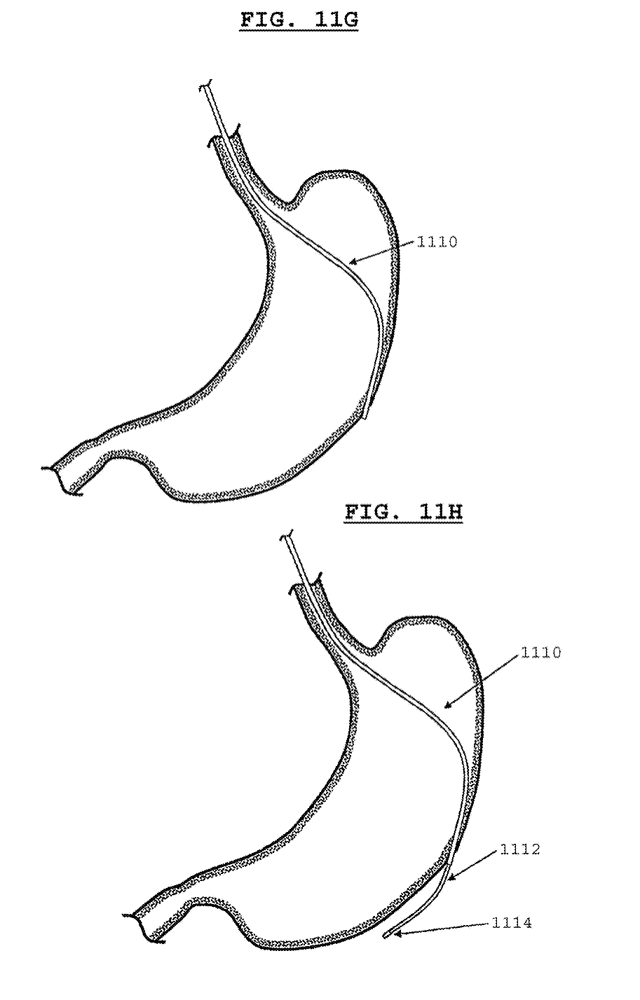

FIGS. 11A-11I depict a method of forming a tract in (e.g., through) stomach tissue. It should be understood that just the distal portion of the device is shown in these figures, and that this method may be used to form tissue tracts as depicted, whether or not the device is a stand alone device, or is used with a gastroscope or advanced through some other sheathed structure (including instances where the device is back-loaded into the working channel of any type of gastroscope, endoscope, laparoscope, etc., with or without steering, visualization, illumination, etc.). Turning now to FIG. 11A, a device (1100) comprising a suction member (1102) is shown advanced adjacent to tissue, here stomach tissue. In FIG. 11B, vacuum or suction has been turned on, and tissue is drawn against, or pulled into, suction member (1102) as indicated by the arrows in that figure. Next, a tissue-piercing member (1104) (e.g., a needle or other tissue-piercing cannula) is advanced from the device and through the drawn tissue to form a tract in the tissue, as shown in FIG. 11C.

Once the tract has been formed, a guidewire (1106), guide element, or the like may be advanced through the tract (e.g., by advancement through a lumen in the tissue-piercing member), as shown in FIG. 11D, and tissue-piercing member (1104) is withdrawn, as shown in FIG. 11E. A stepped-up dilator (1108) or series of dilators (not shown) may then be advanced over guidewire (1106), as shown in FIG. 11F. In this way, for example, the cross-sectional area of the tract may be expanded or enlarged. After the tract has been expanded, an introducer (1110), which may be part of dilator (1108), can be left in place and used as a conduit for introducing additional tools through the tract, as shown in FIG. 11G. FIG. 11H shows one illustrative method where a tool (1112) having an end effector (as shown, grippers (1114), although other appropriate end effectors may be used) has been advanced through introducer (1110) for use in a procedure. Any number or type of tools may be advanced through the introducer in this way. After the procedure has been performed, the tools and introducer are removed, leaving tract (1116) to self-seal (FIG. 1I). Of course, sealing may be enhanced by any suitable additional mechanism (e.g., via mechanical pressure, ultrasound, one or more closure devices, and the like).

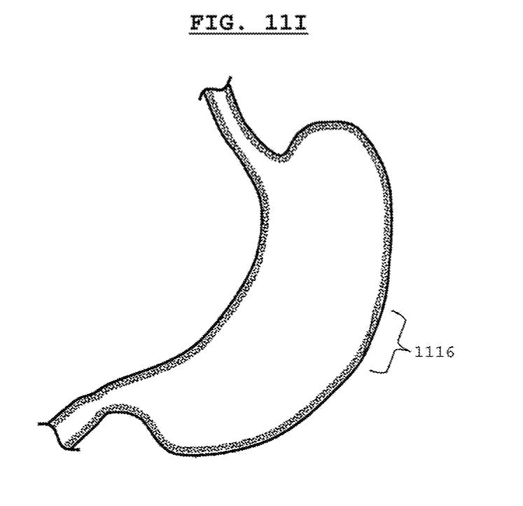

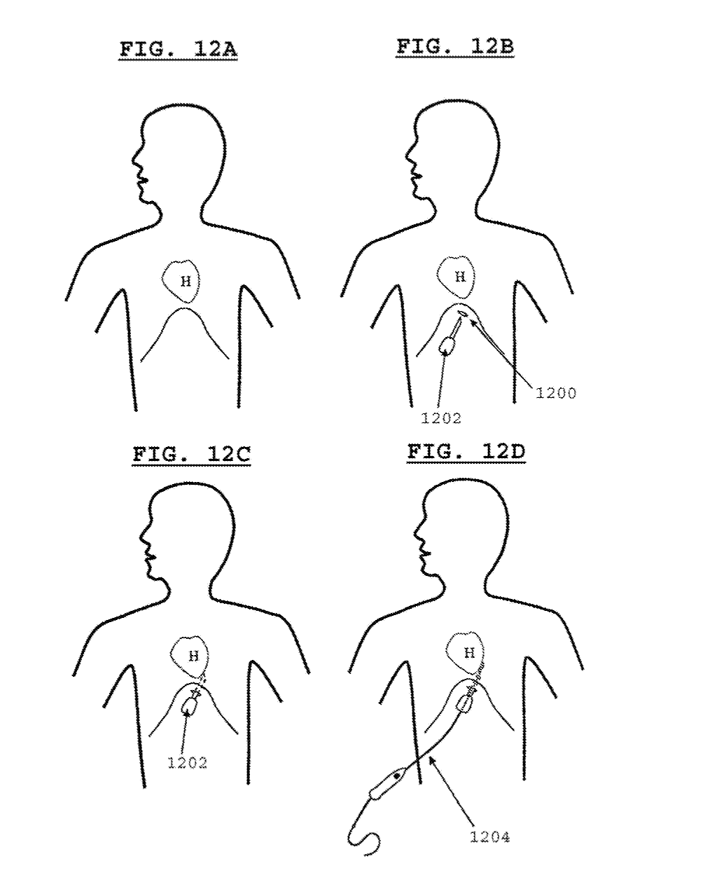

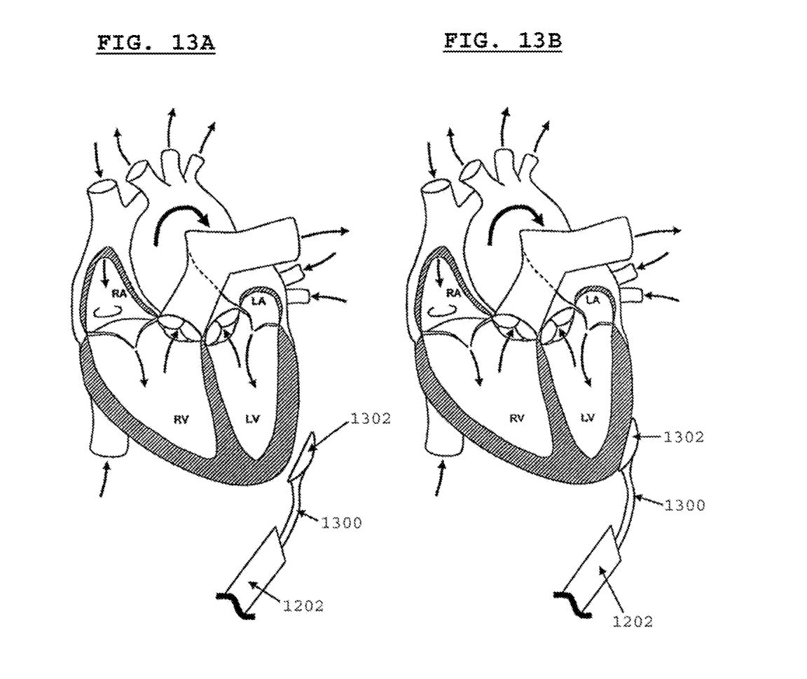

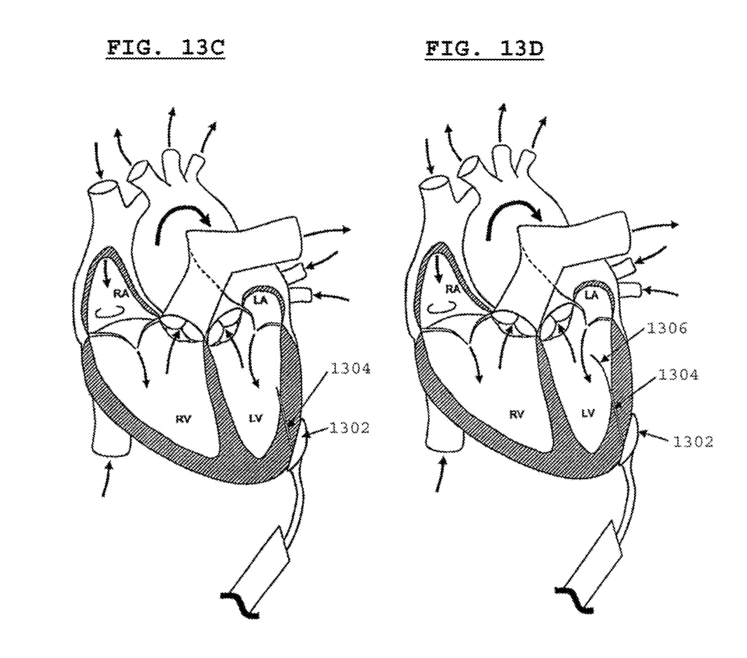

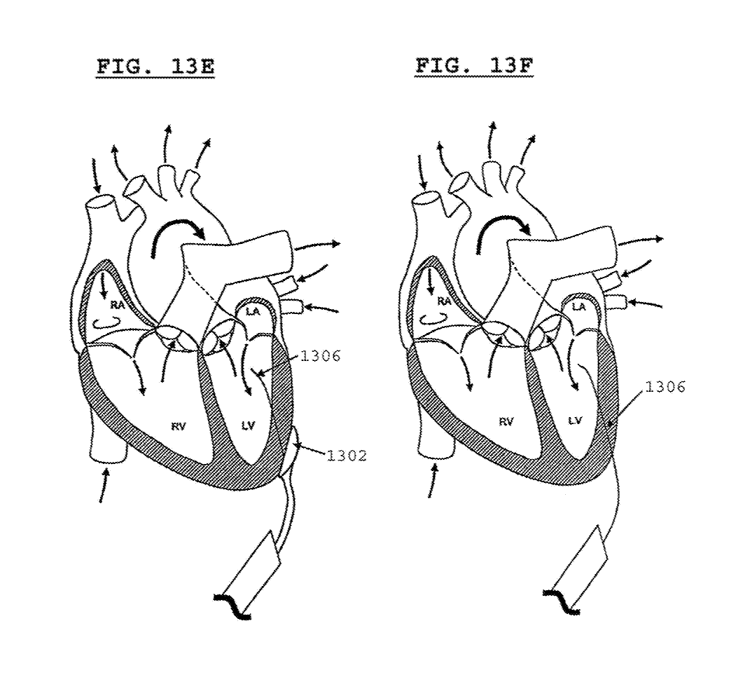

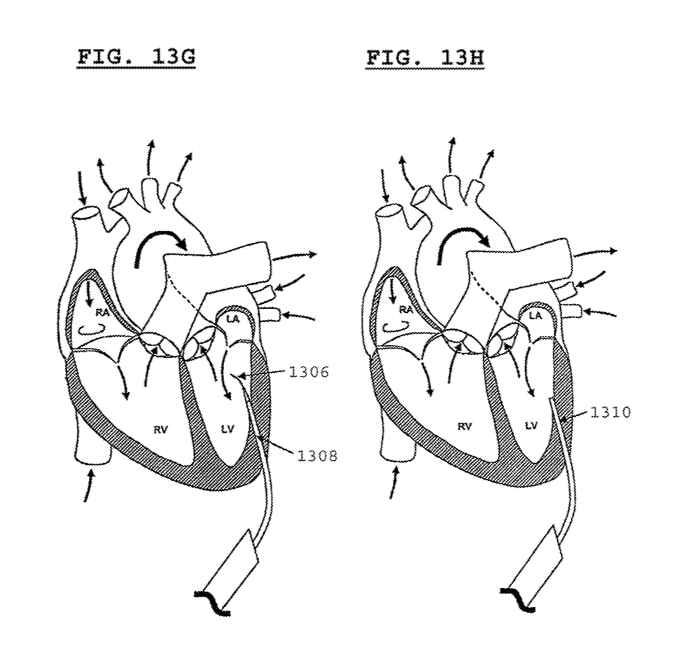

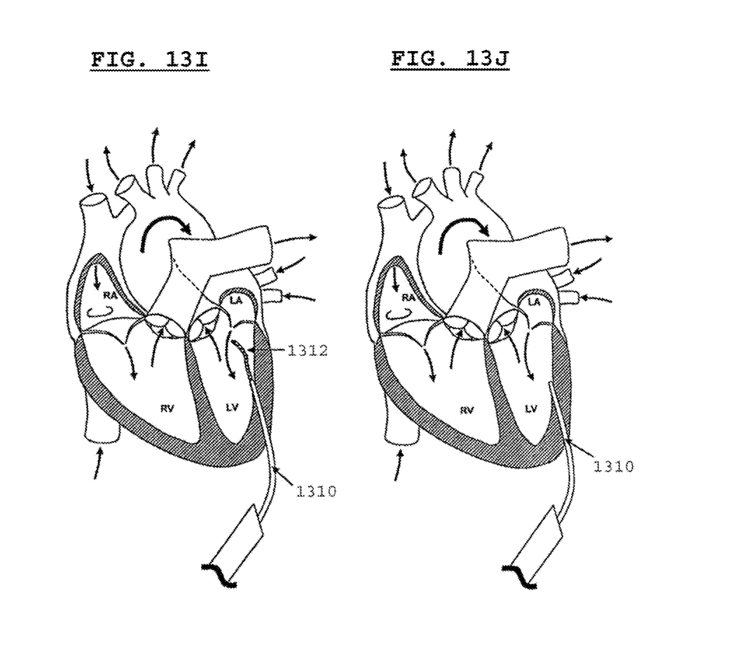

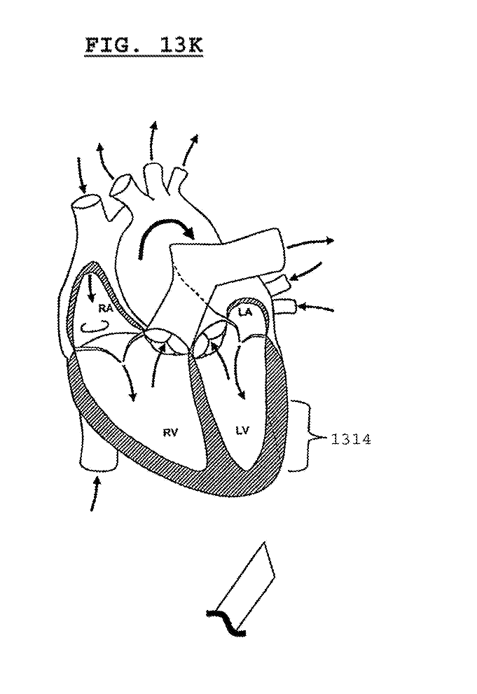

Tracts may also be formed in tissue in one or more other regions of the body. For example, FIG. 12A-12D depict one method of advancing a device described herein into the pericardial space in order to form a tract through tissue of a heart (H). As shown in the figures, an incision (1200) may be made (e.g., sub-xyphoid, etc.) and a port (1202) placed therethrough to provide for suitable delivery or exchange of tools therethrough. Once port (1202) has been placed, any of the devices (1204) described here may be placed through port (1202) to form a tract in or through tissue of heart (H), as will be described in more detail with reference to FIGS. 13A-13K.

Turning now to FIG. 13A, a device (1300) comprising a suction member (1302) is advanced adjacent to heart tissue. The device may be advanced adjacent to heart tissue in any suitable fashion (e.g., through port (1202) described above). Vacuum or suction may then be applied to draw heart tissue against or into suction member (1302), as shown in FIG. 13B. Next, a tissue-piercing member directing device (not shown) may be used to advance a tissue-piercing member (1304) from the device (e.g., through the suction member) and through the drawn tissue to form a tissue tract, as shown in FIG. 13C. A guidewire (1306) or other suitable guide element may then be advanced through the tract (e.g., by advancing through a lumen in the tissue-piercing member (1304), as shown in FIG. 13D). Tissue-piercing member (1304) and device (1300) may then be removed, as shown by FIGS. 13E and 13F, respectively.

A stepped-up dilator (1308) or series of dilators (not shown) may then be advanced over guidewire (1306), as shown in FIG. 13G. In this way, for example, the cross-sectional area of the tract may be expanded or enlarged. After the tract has been expanded, an introducer (1310), which may be part of dilator (1308), can be left in place and used as a conduit for introducing additional tools through the tract, as shown in FIG. 13H. FIG. 13I shows one illustrative method where a tool (1312) has been advanced through introducer (1310) for use in a procedure. Here left ventricular access has been accomplished, and therefore, use of these methods in conjunction with repair or replacement of the aortic or mitral valve may find particular utility. Any number or type of tools may be advanced through the introducer in this way. After the procedure has been performed, the tools and introducer are removed, leaving tract (1314) to self-seal, as shown by FIGS. 13J and 13K. Of course, sealing may be enhanced by any suitable additional mechanism (e.g., via mechanical pressure, ultrasound, one or more closure devices, and the like).

While certain variations of devices have been shown and described, tissue- piercing member directing devices may form a component of one or more other types of devices. For example, a tissue-piercing member directing device may be a component of a clamping device. The clamping device may comprise clamping arms that clamp tissue and secure it such that a tissue-piercing member can pierce through a specific section of the tissue. Thus, the clamping device, in conjunction with the tissue-piercing member directing device, may provide relatively predictable deployment and advancement of the tissue-piercing member through tissue. Clamping devices are described, for example, in U.S. patent application Ser. No. 12/507,043 (published as US 2010/0016810 A1), which was previously incorporated herein by reference in its entirety. In some variations, a device may include both clamping and suction features.

In certain variations, a device may include one or more inflatable members that may help to position tissue for piercing by a tissue-piercing member. For example, the inflatable member or members may position the tissue so that the tissue-piercing member enters the tissue at a specific angle. In addition to helping position the tissue, the inflatable member or members may help to stabilize the device during use (e.g., by temporarily anchoring the device at the target site). For example, when used to form tracts in a vessel wall, the inflatable member or members may contact opposing lumen wall surfaces of the vessel. This may help to prevent the device from slipping or otherwise becoming displaced or moved out of position. Inflatable members may be spherical, donut-shaped, etc. Moreover, in some variations, a device may comprise multiple inflatable members having different sizes and/or shapes. While inflatable members have been described, any suitable expandable region may be employed including, without limitation, hoops or rings (including, e.g., multi-wire hoops), and stents or stent-like structures.

In some variations, one or more of the devices and/or methods described here may be used to form one or more tracts in rotated tissue. For example, a method may comprise positioning a device adjacent a portion of a tissue wall, rotating the portion of the tissue wall (e.g., using the device), and advancing a tissue-piercing member through the rotated tissue to form the tract. The rotating may help to position the tissue-piercing member relative to the tissue wall. The tissue may be rotated in either direction about a tissue circumference (e.g., from 0° to 360°, from 0° to 180°, from 0° to 45°, from 45° to 90°, etc.). However, the tissue need not be rotated a significant amount (e.g., the tissue may be rotated 1°, 5°, 10°, 15°, etc.) and the entire tissue thickness need not be rotated.

In some variations, a portion of tissue may only be rotated once, while in other variations, it may be rotated multiple times (e.g., in the same direction or in different directions). Rotation of tissue prior to and/or during tract formation may be useful to effect a desirable tissue-piercing member location, which may in turn be useful for forming a tract having suitable thicknesses of tissue on either side. This may help ensure that the tract is robust enough to withstand repetitive insertion of various tools. In addition, having sufficient tissue thickness on either side of the tract may help the tract seal more quickly. Initial positioning of the tissue-piercing member away from one or more surfaces of the tissue wall may also help with the formation of a longer tract, which may be useful in ensuring more rapid sealing. The portion of tissue may alternatively or additionally be manipulated in one or more other ways. For example, the portion of tissue may be tented. Methods of manipulating tissue and/or applying a vacuum to tissue are described, for example, in U.S. patent application Ser. No. 11/873,957 (published as US 2009/0105744 A1) and U.S. patent application Ser. No. 12/507,038 (published as US 2010/0016786 A1), both of which were previously incorporated herein by reference in their entirety.

Some variations of the devices described here may comprise one or more heating elements, electrodes, and/or sensors (e.g., Doppler, temperature sensors, pressure sensors, nerve sensors, blood flow sensors, ultrasound sensors, etc.), one or more drug delivery ports along a surface thereof, one or more radiopaque markers to facilitate visualization, or the like. As an example, in some variations, a device may comprise one or more radiopaque materials (e.g., in one or more portions of the device) that may be used to help monitor tract formation. For example, a tissue-piercing member may be made of one or more radiopaque materials or may include radiopaque markings that render the tissue-piercing member visible under X-ray fluoroscopy. In certain variations in which a device comprises one or more sensors, the device may be used to sense at least one useful parameter, such as temperature, pressure, tissue identification or location (e.g., nerves or various anatomical structures), and/or blood flow within a vessel. For example, if the parameter is blood flow within a vessel, the device may be repositioned if blood flow within a vessel is detected.

In some variations, the devices may comprise one or more energy applicators, and may be used to apply energy to tissue. This may, for example, help to seal the tissue. The energy may come from any suitable energy source (e.g., energy selected from the group consisting of ultrasound, radiofrequency (RF), light, magnetic, or combinations thereof). Additionally, certain variations of the devices may comprise one or more cameras (e.g., to facilitate direct visualization). The camera or cameras may or may not have a corresponding light or illumination source, and may be included at any suitable location on a device.

In some variations, a component of a device may, for example, include one or more relatively soft features for contacting a skin surface. As an example, a component of a device may include an inflatable member, such as a relatively soft balloon, that contacts a skin surface when the device is in use. Alternatively or additionally, a component of a device may comprise one or more springs that contact a skin surface when the device is in use (e.g., to provide sufficient tension against the skin surface for isolating a portion of tissue).

In some variations, one or more tracts may be formed in a tissue having one or more irregular tissue surfaces. The irregular surfaces may be in the form of, for example, undulations, bends, curves, recesses, protrusions, any combination of these, or the like. Methods of forming tracts in irregular tissue surfaces are described, for example, in U.S. patent application Ser. No. 11/873,957 (published as US 2009/0105744 A1), which was previously incorporated herein by reference in its entirety.

In some variations, kits may incorporate one or more of the devices and/or device components described here. In certain variations, the kits may include one or more of the devices for forming a tract through tissue described here, one or more of the device components described here (e.g., tissue-piercing members), and/or one or more additional tools. For example, the tools may be those that are advanced through the tract during the performance of a procedure (e.g., guidewires, scissors, grippers, ligation instruments, etc.), one or more supplemental tools for aiding in closure (e.g., an energy delivering device, a closure device, and the like), one or more tools for aiding in the procedure (e.g., gastroscope, endoscope, cameras, light sources, etc.), combinations thereof, and the like. Of course, instructions for use may also be provided with the kits.

While the devices, methods, and kits have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the appended claims. As an example, while tissue-piercing member directing devices that are coupled to tissue-piercing members have been described, in some variations, a tissue-piercing member directing device may be used to deliver a tissue-piercing member that is not coupled to the body of the tissue-piercing member directing device.

What is claimed is:

1. A device for forming a tract in tissue comprising:
a housing comprising at least one groove; and
at least one tissue-piercing member coupled to the housing and comprising a distal end having a tissue-piercing tip,
wherein the at least one tissue-piercing member is movable relative to the housing along a path predetermined by the configuration of the at least one groove.

2. The device of claim 1, further comprising a tissue-piercing member directing device that couples the at least one tissue-piercing member to the housing.

3. The device of claim 2, wherein the tissue-piercing member directing device comprises at least one portion that is slidably disposed within the at least one groove.

4. The device of claim 2, wherein the tissue-piercing member directing device comprises a body and at least one protruding member protruding therefrom, wherein the at least one protruding member is slidably disposed within the at least one groove.

5. The device of claim 1, further comprising at least one tensioning member.

6. The device of claim 5, wherein the at least one tensioning member comprises at least one cable.

7. The device of claim 5, further comprising a rotatable member coupled to or integral with the housing, wherein the at least one tensioning member is coupled to the rotatable member.

8. The device of claim 7, wherein the rotatable member comprises a pulley, and the at least one tensioning member is wound around the pulley.

9. The device of claim 5, wherein proximal translation of the at least one tensioning member results in distal translation of the at least one tissue-piercing member.

10. The device of claim 1, wherein the at least one tissue-piercing member is coupled to the housing at a pivoting point.

11. The device of claim 10, further comprising at least one tensioning member that is coupled to the housing and to the at least one tissue-piercing member, and that is manipulatable to cause the at least one tissue-piercing member to pivot about the pivoting point.

12. The device of claim 1, wherein the housing comprises at least two grooves therein.

13. The device of claim 12, further comprising a tubular member positioned between at least two pins that are slidably disposed within the at least two grooves.

14. The device of claim 13, wherein a portion of the at least one tissue-piercing member is disposed within the tubular member.

15. The device of claim 1, further comprising a tubular member, wherein a portion of the at least one tissue-piercing member is disposed within the tubular member.

16. The device of claim 15, wherein the tubular member is articulatable relative to the housing.

17. The device of claim 1, wherein the housing comprises at least one indented portion.

18. The device of claim 17, wherein the device further comprises a vacuum port located in the at least one indented portion.

19. The device of claim 17, wherein the housing further comprises at least one hook or barb protruding from the at least one indented portion.

20. The device of claim 1, wherein the at least one tissue-piercing member comprises at least one needle.

21. A method for forming a tract in tissue comprising:
positioning a tract-forming device adjacent the tissue, the tract-forming device comprising a housing and at least one tissue-piercing member coupled to the housing; and
advancing the at least one tissue-piercing member along a predetermined path defined by at least one groove in the housing.

22. The method of claim 21, further comprising advancing the at least one tissue-piercing member into the tissue.

23. The method of claim 22, wherein formation of the tract requires advancement only of the at least one tissue-piercing member through the tissue, and wherein the tract is self-sealing.

24. The method of claim 21, wherein the method comprises forming a single tract in the tissue.

25. The method of claim 24, wherein the single tract is self-sealing.

26. The method of claim 21, wherein the predetermined path is curvilinear.

27. The method of claim 21, wherein the tract-forming device further comprises a tensioning member coupled to the at least one tissue-piercing member and the housing, and wherein the method comprises manipulating the tensioning member to advance the at least one tissue-piercing member along the predetermined path.

28. The method of claim 27, wherein manipulating the tensioning member comprises proximally withdrawing the tensioning member.

29. The method of claim 21, further comprising applying a vacuum to the tissue.

30. The method of claim 29, further comprising advancing the at least one tissue-piercing member into the tissue after applying the vacuum to the tissue.

31. The method of claim 21, wherein the tissue comprises an organ.

32. The method of claim 31, wherein the organ is selected from the group consisting of an organ of the cardiovascular system, an organ of the digestive system, an organ of the respiratory system, an organ of the excretory system, an organ of the reproductive system, and an organ of the nervous system.

33. The method of claim 31, wherein the organ is an organ of the cardiovascular system.

34. The method of claim 33, wherein the organ is an artery.

35. The method of claim 21, further comprising withdrawing the at least one tissue-piercing member from the tissue, wherein the tract self-seals after the at least one tissue-piercing member has been withdrawn.

36. The method of claim 35, wherein the tract self-seals within 15 minutes or less.

37. The method of claim 35, wherein the tract self-seals within 10 minutes or less.

38. The method of claim 35, wherein the tract self-seals within 5 minutes or less.

39. The method of claim 21, further comprising advancing one or more tools through the tract.

40. A method for forming a tract in a vessel wall comprising:
positioning a tract-forming device adjacent the vessel wall; and
advancing at least one tissue-piercing member along a predetermined path of the tract-forming device to form a tract in the vessel wall, wherein a portion of the tract traverses the vessel wall substantially parallel to a longitudinal axis of the vessel wall.

41. The method of claim 40, wherein formation of the tract requires advancement only of the at least one tissue-piercing member through the vessel wall, and wherein the tract is self-sealing.

42. The method of claim 40, wherein the method comprises forming a single tract in the vessel wall.

43. The method of claim 42, wherein the single tract is self-sealing.

44. The method of claim 40, wherein the tract-forming device comprises a housing that remains in substantially the same position while the at least one tissue-piercing member forms the tract in the vessel wall.

45. The method of claim 40, wherein the tract-forming device comprises a housing and a tensioning member coupled to both the at least one tissue-piercing member and the housing, and wherein the method comprises manipulating the tensioning member to advance the at least one tissue-piercing member along the predetermined path.

46. The method of claim 45, wherein manipulating the tensioning member comprises proximally withdrawing the tensioning member.

47. The method of claim 40, wherein the vessel is an artery.

48. The method of claim 40, further comprising withdrawing the at least one tissue-piercing member from the vessel wall, wherein the tract self-seals after the at least one tissue-piercing member has been withdrawn.

49. The method of claim 48, wherein the tract self-seals within 15 minutes or less.

50. The method of claim 48, wherein the tract self-seals within 10 minutes or less.

51. The method of claim 48, wherein the tract self-seals within 5 minutes or less.

52. The method of claim 40, further comprising advancing one or more tools through the tract.

53. A method for forming a tract in a vessel wall comprising:
positioning a tract-forming device adjacent the vessel wall; and
advancing at least one tissue-piercing member along a predetermined path of the tract-forming device to form a tract in the vessel wall, wherein the tract forms an angle of less than or equal to about 30° with respect to a longitudinal axis of the vessel wall.

54. The method of claim 53, wherein the tract forms an angle of less than or equal to about 19° with respect to a longitudinal axis of the vessel wall.

55. The method of claim 53, wherein the tract forms an angle of less than or equal to about 15° with respect to a longitudinal axis of the vessel wall.

56. The method of claim 53, wherein the tract forms an angle of less than or equal to about 10° with respect to a longitudinal axis of the vessel wall.

57. The method of claim 53, wherein the tract forms an angle of less than or equal to about 5° with respect to a longitudinal axis of the vessel wall.

58. The method of claim 53, wherein the tract forms an angle of about 1° to about 30° with respect to a longitudinal axis of the vessel wall.

59. The method of claim 53, wherein the tract forms an angle of about 1° to about 19° with respect to a longitudinal axis of the vessel wall.

60. The method of claim 53, wherein the tract forms an angle of about 1° to about 15° with respect to a longitudinal axis of the vessel wall.

61. The method of claim 53, wherein the tract forms an angle of about 1° to about 10° with respect to a longitudinal axis of the vessel wall.

62. The method of claim 53, wherein the tract forms an angle of about 1° to about 5° with respect to a longitudinal axis of the vessel wall.

63. The method of claim 53, wherein the tract forms an angle of about 5° to about 15° with respect to a longitudinal axis of the vessel wall.

64. The method of claim 53, wherein the tract forms an angle of about 5° to about 10° with respect to a longitudinal axis of the vessel wall.

65. The method of claim 53, wherein formation of the tract requires advancement only of the at least one tissue-piercing member through the vessel wall, and wherein the tract is self-sealing.

66. The method of claim 53, wherein the method comprises forming a single tract in the vessel wall.

67. The method of claim 66, wherein the single tract is self-sealing.

68. The method of claim 53, wherein the tract-forming device comprises a housing that remains in substantially the same position while the at least one tissue-piercing member forms the tract in the vessel wall.

69. The method of claim 53, wherein the tract-forming device comprises a housing and a tensioning member coupled to both the at least one tissue-piercing member and the housing, and wherein the method comprises manipulating the tensioning member to advance the at least one tissue-piercing member along the predetermined path.

70. The method of claim 69, wherein manipulating the tensioning member comprises proximally withdrawing the tensioning member.

71. The method of claim 53, wherein the vessel is an artery.

72. The method of claim 53, further comprising withdrawing the at least one tissue-piercing member from the vessel wall, wherein the tract self-seals after the at least one tissue-piercing member has been withdrawn.
